(19) United States
(12) Patent Application Publication
(10) Pub. No.: US 2009/0326049 A1
(43) Pub. Date: Dec. 31, 2009

(54) BLOCKING OLIGOS FOR INHIBITION OF MICRORNA AND SIRNA ACTIVITY AND USES THEREOF

(76) Inventors: Alexander Aristarkhov, Chestnut Hill, MA (US); Søren Morgenthaler Echwald, Humlebaek (DK)

Correspondence Address: CLARK & ELBING LLP, 101 FEDERAL STREET, BOSTON, MA, 02110, US

(21) Appl. No.: 12/417,397

(22) Filed: Apr. 2, 2009

(30) Foreign Application Priority Data
Apr. 4, 2008 (DK) PA200800497

(51) Int. Cl. A61K 31/7088; C12Q 1/68; C07H 21/00; C12N 5/06

(52) U.S. Cl. 514/44 R; 435/6; 536/22.1; 435/375

(57) ABSTRACT

The present invention relates to methods of identifying sites in the 3′- and/or 5′-UTR of mRNA involved in the binding of miRNA and/or siRNA to their target sites and nucleic acids designed to prevent the binding of endogenous or exogenous miRNA and/or siRNA to their target mRNA and uses thereof.

### CROSS-REFERENCE TO RELATED APPLICATIONS

This application claims the benefit of Danish Patent Application Number PA 200800497, filed Apr. 4, 2008.

The present invention relates to methods of identifying sites in the 3′- and/or 5′-UTR of mRNA involved in the binding of miRNA and/or siRNA to their target sites and nucleic acids designed to prevent the binding of endogenous or exogenous microRNA and/or siRNA to their target mRNA and uses thereof.

### BACKGROUND OF THE INVENTION

The present invention relates to the study and modulation of the effect of small RNAs on target nucleotide sequences in a wide variety of nucleic acid samples. More specifically the present invention is directed to methods for identifying sites involved in the binding of small RNAs and for designing oligonucleotides that are useful for preventing the binding of endogenous or exogenous microRNA and/or siRNA especially to RNA target sequences, such as microRNA and/or siRNA target sites.

### MicroRNAs

The expanding inventory of international sequence databases and the concomitant sequencing of nearly 200 genomes representing all three domains of life—bacteria, archea, and eukaryota—have been the primary drivers in the process of deconstructing living organisms into comprehensive molecular catalogs of genes, transcripts, and proteins. The importance of the genetic variation within a single species has become apparent, extending beyond the completion of genetic blueprints of several important genomes, culminating in the publication of the working draft of the human genome sequence in 2001 (Lander, Linton, Birren et al., 2001 Nature 409: 860-921; Venter, Adams, Myers et al., 2001 Science 291: 1304-1351; Sachidanandam, Weissman, Schmidt et al., 2001 Nature 409: 928-933). On the other hand, the increasing number of detailed, large-scale molecular analyses of transcription originating from the human and mouse genomes along with the recent identification of several types of non-protein-coding RNAs, such as small nucleolar RNAs, siRNAs, microRNAs and antisense RNAs, indicate that the transcriptomes of higher eukaryotes, are much more complex than originally anticipated (Wong et al., 2001, Genome Research 11: 1975-1977; Kampa et al. 2004, Genome Research 14: 331-342).

As a result of the Central Dogma: ‘DNA makes RNA, and RNA makes protein’, RNAs have been considered as simple molecules that just translate the genetic information into protein. Recently, it has been estimated that although most of the genome is transcribed, almost 97% of the genome does not encode proteins in higher eukaryotes, but putative, non-coding RNAs (Wong et al. 2001, Genome Research 11: 1975-1977). The non-coding RNAs (ncRNAs) appear to be particularly well suited for regulatory roles that require highly specific nucleic acid recognition. Therefore, the view of RNA is rapidly changing from the merely informational molecule to comprise a wide variety of structural, informational and catalytic molecules in the cell.

Recently, a large number of small non-coding RNA genes have been identified and designated as microRNAs (miRNAs) (for review, see Ke et al. 2003, Curr. Opin. Chem. Biol. 7:516-523). The first miRNAs to be discovered were the lin-4 and let-7 that are heterochronic switching genes essential for the normal temporal control of diverse developmental events (Lee et al. 1993, Cell 75:843-854; Reinhart et al. 2000, Nature 403: 901-906) in the roundworm C. elegans. miRNAs have been evolutionarily conserved over a wide range of species and exhibit diversity in expression profiles, suggesting that they occupy a wide variety of regulatory functions and exert significant effects on cell growth and development (Ke et al. 2003, Curr. Opin. Chem. Biol. 7:516-523). Recent work has shown that miRNAs can regulate gene expression at many levels, representing a novel gene regulatory mechanism and supporting the idea that RNA is capable of performing similar regulatory roles as proteins. Understanding this RNA-based regulation will help us to understand the complexity of the genome in higher eukaryotes as well as understand the complex gene regulatory networks.

miRNAs are 19-25 nucleotide (nt) RNAs that are processed from longer endogenous hairpin transcripts (Ambros et al. 2003, RNA 9: 277-279). To date more than 5000 miRNAs have been identified in humans, worms, fruit flies and plants according to the miRNA registry database release 10.1 in December 2007, hosted by Sanger Institute, UK, and many miRNAs that correspond to putative genes have also been identified. Some miRNAs have multiple loci in the genome (Reinhart et al. 2002, Genes Dev. 16: 1616-1626) and occasionally, several miRNA genes are arranged in tandem clusters (Lagos-Quintana et al. 2001, Science 294: 853-858). The fact that many of the miRNAs reported to date have been isolated just once suggests that many new miRNAs will be discovered in the future. A recent in-depth transcriptional analysis of the human chromosomes 21 and 22 found that 49% of the observed transcription was outside of any known annotation, and furthermore, that these novel transcripts were both coding and non-coding RNAs (Kampa et al. 2004, Genome Research 14: 331-342). The identified miRNAs to date represent most likely the tip of the iceberg, and the number of miRNAs might turn out to be very large.

The combined characteristics of miRNAs characterized to date (Ke et al. 2003, Curr. Opin. Chem. Biol. 7:516-523; Lee et al. 1993, Cell 75:843-854; Reinhart et al. 2000, Nature 403: 901-906) can be summarized as:
1. miRNAs are single-stranded RNAs of about 19-25 nt that regulate the expression, stability, and/or translation into protein of complementary messenger RNAs
2. They are cleaved from a longer endogenous double-stranded hairpin precursor by the enzyme Dicer.
3. miRNAs match precisely the genomic regions that can potentially encode precursor RNAs in the form of double-stranded hairpins.
4. miRNAs and their predicted precursor secondary structures are phylogenetically conserved.

Several lines of evidence suggest that the enzymes Dicer and Argonaute are crucial participants in miRNA biosynthesis, maturation, and function (Grishok et al. 2001, Cell 106: 23-24). Mutations in genes required for miRNA biosynthesis lead to genetic developmental defects, which are, at least in part, derived from the role of generating miRNAs. The current view is that miRNAs are cleaved by Dicer from the hairpin precursor in the form of duplex, initially with 2 or 3 nt overhangs in the 3′ ends, and are termed pre-miRNAs. Cofactors join the pre-miRNP and unwind the pre-miRNAs into single-stranded miRNAs, and pre-miRNP is then transformed to miRNP. miRNAs can recognize regulatory targets while part of the miRNP complex. There are several similarities between miRNP and the RNA-induced silencing complex, RISC (involved in RNA interference by siRNAs), including similar sizes and both containing RNA helicase and the PPD proteins. It has therefore been proposed that miRNP and RISC are the same RNP with multiple functions (Ke et al. 2003, Curr. Opin. Chem. Biol. 7:516-523).

Different effectors direct miRNAs into diverse pathways. The structure of pre-miRNAs is consistent with the observation that 22 nt RNA duplexes with 2 or 3 nt overhangs at the 3′ ends are beneficial for reconstitution of the protein complex and might be required for high affinity binding of the short RNA duplex to the protein components (for review, see Ke et al., 2003, Curr. Opin. Chem. Biol. 7:516-523).

Growing evidence suggests that miRNAs play crucial roles in eukaryotic gene regulation. The first miRNA genes to be discovered, lin-4 and let-7, base-pair incompletely to repeated elements in the 3′ untranslated regions (UTRs) of other heterochronic genes, and regulate the translation directly and negatively by antisense RNA-RNA interaction (Lee et al. 1993, Cell 75:843-854; Reinhart et al. 2000, Nature 403: 901-906). Other miRNAs are thought to interact with target mRNAs by limited complementary and suppressed translation as well (Lagos-Quintana et al., 2001, Science 294: 853-858; Lee and Ambros 2001, Science 294: 858-862). Many studies have shown, however, that given a perfect complementarity between miRNAs and their target RNA, could lead to target RNA degradation rather than inhibit translation (Hutvagner and Zamore 2002, Science 297: 2056-2060), suggesting that the degree of complementarity determines their functions. A recent publication indicated that miRNA can not only inhibit but also increase translation from target mRNA (Vasudevan et al., Science 318; 1931-4, 2007).

By identifying sequences with near complementarity, several targets have been predicted, most of which appear to be potential transcriptional factors that are crucial in cell growth and development. The high percentage of predicted miRNA targets acting as developmental regulators and the conservation of target sites suggest that miRNAs are involved in a wide range of organism development and behaviour and cell fate decisions (for review, see Ke et al. 2003, Curr. Opin. Chem. Biol. 7:516-523). For example, John et al. 2004 (PLoS Biology 2: e363) used known mammalian miRNAs to scan the 3′ untranslated regions (UTRs) from human, mouse and rat genomes for potential miRNA target sites using a scanning algorithm based on sequence complementarity between the mature miRNA and the target site, binding energy of the miRNA:mRNA duplex and evolutionary conservation. They identified a total of 2307 target mRNAs conserved across the mammals with more than one target site at 90% conservation of target site sequence and 660 target genes at 100% conservation level. Scanning of the two fish genomes; Danio rerio (zebrafish) and Fugu rubripes (Fugu) identified 1000 target genes with two or more conserved miRNA sites between the two fish species (John et al. 2004 PLoS Biology 2: e363). Among the predicted targets, particularly interesting groups included mRNA encoding transcription factors, components of the miRNA machinery, other proteins involved in the translational regulation as well as components of the ubiquitin machinery. Wang et al. 2004 (Genome Biology 5:R65) have developed and applied a computational algorithm to predict 95 Arabidopsis thaliana miRNAs, which included 12 known ones and 83 new miRNAs. The 83 new miRNAs were found to be conserved with more than 90% sequence identity between the Arabidopsis and rice genomes. Using the Smith-Waterman nucleotide-alignment algorithm to predict mRNA targets for the 83 new miRNAs and by focusing on target sites that were conserved in both Arabidopsis and rice, Wang et al. 2004 (Genome Biology 5:R65) predicted 371 mRNA targets with an average of 4.8 targets per miRNA. A large proportion of these mRNA targets encoded proteins with transcription regulatory activity.

Multiple miRNA target sites have been shown to be associated with greater mRNA destabilization. Intervening sequences between repeated target sites of 13-35 nt have been shown to be necessary for strong miRNA regulation of miRNA targets and one important context determinant that influences efficacy of miRNA target sites is their proximity to sites for co-expressed miRNAs (Grimson et al., Mol. Cell. 27; 91-105, 2007; Saetrom et al., Nucleic Acids Res. 35(7); 2333-2342, 2007). Cooperative miRNA function implies a mechanism whereby repression can become more sensitive to small changes in miRNA levels. Moreover, cooperativity of sites for coexpressed miRNAs greatly enhances the regulatory effect and utility of combinatorial miRNA expression.

Recent work from Kedde et al. (Cell 131; 1273-1286, 2007) has provided evidence of a RNA binding proteins, Dnd1, capable of mediating an inhibitory effect on miRNA binding to miRNA target sites through uridine-rich regions present in the miRNA-targeted mRNA.

Conserved regions in mRNA different from miRNA target sites may be involved in miRNA binding i.e. by providing a docking platform for modulators of miRNA activity. It has been proposed that such modulators may physically block access of the miRNP complex to an miRNA target site or change the subcellular localization of an mRNA to a compartment out of reach of miRNAs (Ketting, Cell 131; 1226-1227, 2006).

Thus, co-expression of a miRNA and a particular target mRNA in the same cell is not a guarantee of miRNA activity on the target mRNA. The binding of miRNA to target may require the presence of co-factors—such as RNA binding proteins, protein complexes or another RNA with regulatory function. In fact the same mRNA may be targeted by several miRNAs which function cooperatively to inhibit or induce translation, mRNA degradation or trafficking inside cells.

### MiRNAs and Human Disease

Analysis of the genomic location of miRNAs indicates that they play important roles in human development and disease. Several human diseases have already been pinpointed in which miRNAs or their processing machinery might be implicated. One of them is spinal muscular atrophy (SMA), a paediatric neurodegenerative disease caused by reduced protein levels or loss-of-function mutations of the survival of motor neurons (SMN) gene (Paushkin et al. 2002, Curr. Opin. Cell Biol. 14: 305-312). Two proteins (Gemin3 and Gemin4) that are part of the SMN complex are also components of miRNPs, whereas it remains to be seen whether miRNA biogenesis or function is dysregulated in SMA and what effect this has on pathogenesis. Another neurological disease linked to mi/siRNAs is fragile X mental retardation (FXMR) caused by absence or mutations of the fragile X mental retardation protein (FMRP) (Nelson et al. 2003, TIBS 28: 534-540), and there are additional clues that miRNAs might play a role in other neurological diseases. Yet another interesting finding is that the miR-224 gene locus lies within the minimal candidate region of two different neurological diseases: early-onset Parkinsonism and X-linked mental retardation (Dostie et al., 2003, RNA: 9: 180-186). Links between cancer and miRNAs have also been recently described. The most frequent single genetic abnormality in chronic lymphocytic leukaemia (CLL) is a deletion localized to chromosome 13q14 (50% of the cases). A recent study determined that two different miRNA (miR15 and miR16) genes are clustered and located within the intron of LEU2, which lies within the deleted minimal region of the B-cell chronic lymphocytic leukaemia (B-CLL) tumour suppressor locus, and both genes are deleted or down-regulated in the majority of CLL cases (Calin et al. 2002, Proc. Natl. Acad. Sci. U.S.A. 99: 15524-15529). It has been anticipated that connections between miRNAs and human diseases will only strengthen in parallel with the knowledge of miRNAs and the gene networks that they control. Moreover, the understanding of the regulation of RNA-mediated gene expression is leading to the development of novel therapeutic approaches that will be likely to revolutionize the practice of medicine (Nelson et al. 2003, TIBS 28: 534-540).

### Small Interfering RNAs and RNA Interference

Some of the recent attention paid to small RNAs in the size range of 21 to 25 nt is due to the phenomenon RNA interference (RNAi), in which double-stranded RNA leads to the degradation of any RNA that is homologous (Fire et al. 1998, Nature 391: 806-811). RNAi relies on a complex and ancient cellular mechanism that has probably evolved for protection against viral attack and mobile genetic elements. A crucial step in the RNAi mechanism is the generation of short interfering RNAs (siRNAs), double-stranded RNAs that are about 22 nt long each. The siRNAs lead to the degradation of homologous target RNA and the production of more siRNAs against the same target RNA (Lipardi et al. 2001, Cell 107: 297-307). The present view for the mRNA degradation pathway of RNAi is that antiparallel Dicer dimers cleave long double-stranded dsRNAs to form siRNAs in an ATP-dependent manner. The siRNAs are then incorporated in the RNA-induced silencing complex (RISC) and ATP-dependent unwinding of the siRNAs activates RISC (Zhang et al. 2002, EMBO J. 21: 5875-5885; Nykanen et al. 2001, Cell 107: 309-321). The active RISC complex is thus guided to degrade the specific target mRNAs.

One of the perceived advantages using siRNA as a functional genomics tool is its ability to silence genes in a sequence-specific manner. While long double-stranded RNA molecules can be employed to induce RNAi in lower eukaryotes, siRNAs have to be used for gene silencing in mammalian cells in order to prevent the activation of an unspecific interferon response (Elbashir et al., Nature; 411; 494-498, 2001). Gene expression in cell cultures can be conveniently blocked by either transfecting the siRNA into cells (Janowski et al.; Nat. Protoc. 1; 436-443, 2006) or by introducing a vector that can express the siRNA within the cells (Tiscornia et al., Nat. Protoc; 1; 234-240, 2006). A 7 nt complementation between the siRNA and the target site has been found to be sufficient to cause gene silencing and sequences surrounding the siRNA target sites are also important for the silencing effect (Lin et al., Nucleic Acids Res 33(14); 4527-4535, 2005).

For the RNAi pathway to be a useful tool in the research and therapeutic venues, the siRNA, must be designed to be both potent and specific in its targeting of messenger RNA transcripts. Multiple design algorithms have been developed that enhance the selection of highly functional duplexes and the accurate prediction of siRNA target gene knockdown (Naito et al., Nucleic Acid Res. 32; W124-W129, 2004; Jagla et al., RNA 11; 864-872, 2005; Huesken et al. Nat. Biotechnol. 23; 995-1001, 2005). However, less is known about the parameters that contribute to siRNA specificity. Unintended gene modulation can result from lipid delivery reagents and siRNA induction of the innate cellular immunity. A third contributor to unintended gene knockdown is associated with off-targeting.

### Off-Targeting

Off target gene silencing is an RNAi-mediated event that results in changes in the expression of several genes by different mechanisms including global up/down-regulation of genes using high concentrations of siRNA, the induction of an interferon response, miRNA-like translational inhibition and mRNA degradation mediated by partial sequence complementarity. Recent work has indicated that some off-target effects are caused by the siRNAs cooperating with endogenous miRNAs at optimally spaced target sites to down-regulate mRNAs (Saetrom et al., Nucleic Acids Res. 35(7); 2333-2342, 2007). Off-target effects can be mediated by either strand of the siRNA and have been documented to occur when 15 base pairs, and as few as 11 contiguous base pairs, of sequence identity exist between the siRNA and off-target transcript (Jackson et al. Nat. Biotechnol. 21; 635-637; 2003).

As off-targeting can induce measurable phenotypes, including potential toxicity, and problems in data interpretation (Lin et al., Nucleic Acids Res. 33; 4527-4535, 2005), it represents one of the largest impediments for therapeutic and phenotypic screening applications for RNAi.

Comparison of validated off-target data set with in silico predicted off-targets recently showed that overall identity, except for near-perfect matches, does not accurately predict off-targeted genes (Birmingham et al., Nature Methods 3(3); 199-204, 2006). Perfect matches between the hexamer or heptamer seed region (positions 2-7 or 2-8 of the antisense strand) of an siRNA and the 3′ UTR were found to be associated with off-targeting. Nevertheless, only a small percentage of transcripts that contain seed sites are significantly down-regulated by the siRNAs. These results indicate a strong mechanistic parallel between siRNA off-targeting and miRNA-mediated gene regulation and reveal that current protocols used to minimize off-target effects (f.ex. blastn and Smith-Waterman algorithm) have little merit aside from eliminating the most obvious off-targets.

Microarray-based gene expression analysis has previously been used as a method of off-target identification (Jackson et al. Nat. Biotechnol. 21; 635-637; 2003).

Detection and Analysis of miRNAs and siRNAs

The current view that miRNAs may represent a newly discovered, hidden layer of gene regulation has resulted in high interest among researchers around the world in the discovery of miRNAs, their targets and mechanism of action. Detection and analysis of these small RNAs is, however not trivial. Thus, the discovery of more than 1400 miRNAs to date has required taking advantage of their special features. First, the research groups have used the small size of the miRNAs as a primary criterion for isolation and detection. Consequently, standard cDNA libraries would lack miRNAs, primarily because RNAs that small are normally excluded by size selection in the cDNA library construction procedure. Total RNA from fly embryos, worms or HeLa cells have been size fractionated so that only molecules 25 nucleotides or smaller would be captured (Moss 2002, Curr. Biology 12: R138-R140). Synthetic oligomers have then been ligated directly to the RNA pools using T4 RNA ligase. Then the sequences have been reverse-transcribed, amplified by PCR, cloned and sequenced (Moss 2002, Curr. Biology 12: R138-R140). The genome databases have subsequently been queried with the sequences, confirming the origin of the miRNAs from these organisms as well as placing the miRNA genes physically in the context of other genes in the genome. The vast majority of the cloned sequences have been located in intronic regions or between genes, occasionally in clusters, suggesting that the tandemly arranged miRNAs are processed from a single transcript to allow coordinated regulation. Furthermore, the genomic sequences have revealed the fold-back structures of the miRNA precursors (Moss 2002, Curr. Biology 12: R138-R140).

The size and often low level of expression of different miRNAs require the use of sensitive and quantitative analysis tools. Due to their small size of 19-25 nt, the use of quantitative real-time PCR for monitoring expression of mature miRNAs is excluded. Therefore, most miRNA researchers currently use Northern blot analysis combined with polyacrylamide gels to examine expression of both the mature and pre-miRNAs (Reinhart et al. 2000, Nature 403: 901-906; Lagos-Quintana et al. 2001, Science 294: 853-858; Lee and Ambros 2001, Science 294: 862-864). Primer extension has also been used to detect the mature miRNA (Zeng and Cullen 2003, RNA 9: 112-123). The disadvantage of all the gel-based assays (Northern blofting, primer extension, RNase protection assays etc.) as tools for monitoring miRNA expression includes low throughput and poor sensitivity. Consequently, a large amount of total RNA per sample is required for Northern analysis of miRNAs, which is not feasible when the cell or tissue source is limited.

DNA microarrays would appear to be a good alternative to Northern blot analysis to quantify miRNAs in a genome-wide scale, since microarrays have excellent throughput. Krichevsky et al. 2003 used cDNA microarrays to monitor the expression of miRNAs during neuronal development with 5 to 10 μg aliquot of input total RNA as target, but the mature miRNAs had to be separated from the miRNA precursors using micro concentrators prior to microarray hybridizations (Krichevsky et al. 2003, RNA 9: 1274-1281). Liu et al 2004 (Liu et al. 2004, Proc. Natl. Acad. Sci, U.S.A 101:9740-9744) have developed a microarray for expression profiling of 245 human and mouse miRNAs using 40-mer DNA oligonucleotide capture probes. Thomson et al. 2004 (Thomson et al. 2004, Nature Methods 1:1-6) describe the development of a custom oligonucleotide microarray platform for expression profiling of 124 mammalian miRNAs conserved in human and mouse using oligonucleotide capture probes complementary to the mature miRNAs. The microarray was used in expression profiling of the 124 miRNAs in question in different adult mouse tissues and embryonic stages. A similar approach was used by Miska et al. 2004 (Genome Biology 2004; 5:R68) for the development of an oligoarray for expression profiling of 138 mammalian miRNAs, including 68 miRNAs from rat and monkey brains. Yet another approach was taken by Barad et al. 2004 (Genome Research 2004; 14: 2486-2494), who developed a 60-mer oligonucleotide microarray platform for known human mature miRNAs and their precursors. The drawback of all DNA-based oligonucleotide arrays regardless of the capture probe length is the requirement of high concentrations of labelled input target RNA for efficient hybridization and signal generation, low sensitivity for rare and low-abundant miRNAs, and the necessity for post-array validation using more sensitive assays such as real-time quantitative PCR, which is not currently feasible. In addition, at least in some array platforms discrimination of highly homologous miRNA differing by just one or two nucleotides could not be achieved, thus presenting problems in data interpretation, although the 60-mer microarray by Barad et al. 2004 (Genome Research 2004; 14: 2486-2494) appears to have adequate specificity.

A PCR approach has also been used to determine the expression levels of mature miRNAs (Grad et al. 2003, Mol. Cell. 11: 1253-1263). This method is useful to clone miRNAs, but highly impractical for routine miRNA expression profiling, since it involves gel isolation of small RNAs and ligation to linker oligonucleotides. Allawi et al. (2004, RNA 10: 1153-1161) have developed a method for quantification of mature miRNAs using a modified Invader assay. Although apparently sensitive and specific for the mature miRNA, the drawback of the Invader quantification assay is the number of oligonucleotide probes and individual reaction steps needed for the complete assay, which increases the risk of cross-contamination between different assays and samples, especially when high-throughput analyses are desired. Schmittgen et al. (2004, Nucleic Acids Res. 32: e43) describe an alternative method to Northern blot analysis, in which they use real-time PCR assays to quantify the expression of miRNA precursors. The disadvantage of this method is that it only allows quantification of the precursor miRNAs, which does not necessarily reflect the expression levels of mature miRNAs. In order to fully characterize the expression of large numbers of miRNAs, it is necessary to quantify the mature miRNAs, such as those expressed in human disease, where alterations in miRNA biogenesis produce levels of mature miRNAs that are very different from those of the precursor miRNA. For example, the precursors of 26 miRNAs were equally expressed in non-cancerous and cancerous colorectal tissues from patients, whereas the expression of mature human miR143 and miR145 was greatly reduced in cancer tissues compared with non-cancer tissues, suggesting altered processing for specific miRNAs in human disease (Michael et al. 2003, Mol. Cancer. Res. 1: 882-891). On the other hand, recent findings in maize with miR166 and miR165 in Arabidopsis thaliana, indicate that miRNAs act as signals to specify leaf polarity in plants and may even form movable signals that emanate from a signalling centre below the incipient leaf (Juarez et al. 2004, Nature 428: 84-88; Kidner and Martienssen 2004, Nature 428: 81-84).

Most of the miRNA expression studies in animals and plants have utilized Northern blot analysis, tissue-specific small RNA cloning, and expression profiling by microarrays or real-time PCR of the miRNA hairpin precursors, as described above. However, these techniques lack the resolution for addressing the spatial and temporal expression patterns of mature miRNAs. Due to the small size of mature miRNAs, detection of them by standard RNA in situ hybridization has proven difficult to adapt in both plants and vertebrates, even though in situ hybridization has recently been reported in A. thaliana and maize using RNA probes corresponding to the stem-loop precursor miRNAs (Chen et al., 2004, Science 203: 2022-2025; Juarez et al. 2004, Nature 428: 84-88). Brennecke et al. 2003 (Cell 113: 25-36) and Mansfield et al. 2004 (Nature Genetics 36: 1079-83) report on an alternative method in which reporter transgenes, so-called sensors, are designed and generated to detect the presence of a given miRNA in an embryo. Each sensor contains a constitutively expressed reporter gene (e.g. lacZ or green fluorescent protein) harbouring miRNA target sites in its 3′-UTR. Thus, in cells that lack the miRNA in question, the transgene RNA is stable allowing detection of the reporter, whereas cells expressing the miRNA, the sensor mRNA is targeted for degradation by the RNAi pathway. Although sensitive, this approach is time-consuming since it requires generation of the expression constructs and transgenes. Furthermore, the sensor-based technique detects the spatiotemporal miRNA expression patterns via an indirect method as opposed to direct in situ hybridization of the mature miRNAs.

The large number of miRNAs along with their small size makes it difficult to create loss-of-function mutants for functional genomics analyses. Another potential problem is that many miRNA genes are present in several copies per genome occurring in different loci, which makes it even more difficult to obtain mutant phenotypes. Boutla et al. 2003 (Nucleic Acids Research 31: 4973-4980) describe the use of DNA antisense oligonucleotides complementary to 11 different miRNAs in Drosophila as well as their use to inactivate the miRNAs by injecting the DNA oligonucleotides into fly emryos. Of the 11 DNA antisense oligonucleotides, only 4 constructs showed severe interference with normal development, while the remaining 7 oligonucleotides didn't show any phenotypes presumably due to their inability to inhibit the miRNA in question. Thus, the success rate for using DNA antisense oligonucleotides to inhibit miRNA function would most likely be too low to allow functional analyses of miRNAs on a larger, genomic scale. An alternative approach to this has been reported by Hutvagner et al. 2004 (PLoS Biology 2: 1-11), in which 2′-O-methyl antisense oligonucleotides could be used as potent and irreversible inhibitors of miRNA and siRNA function in vitro and in vivo in Drosophila and C. elegans, thereby inducing a loss-of-function phenotype. A drawback of this method is the need of high 2′-O-methyl oligonucleotide concentrations (100 micromolar) in transfection and injection experiments, which may be toxic to the animal.

In conclusion, a challenge in functional analysis and therapeutic modulation of the mature miRNAs as well as siRNAs using currently available methods is the ability of miRNAs and siRNAs to interact with target nucleic acids through imperfect target site recognition and hence for each miRNA and siRNA to target multiple target nucleotides in an undesired manner. Current methods for inhibition of miRNA activity such as the anti-miRNA oligonucleotides (Weiler et al, Gene Ther 13; 496-502, 2005), the ‘antagomirs’ (Krutzfeldt et al., Nature 438; 685-689, 2005) and miRNA ‘sponges’ (Ebert et al., Nature Methods, 4(9); 721-726, 2007) inhibit the activity of the targeted miRNA by a reduction in miRNA:target interaction over a broad range. The present invention provides methods for identifying sites in the 3′ or 5′ UTR of a particular mRNA involved in regulating the binding of a miRNA and/or siRNA to a miRNA and/or siRNA target site within the mRNA and cofactors of miRNA activity binding to the identified sites. The present invention also provides the design and development of novel oligonucleotide compounds hybridising to a naturally occurring nucleotide sequence upstream (5′) and downstream (3′) of a miRNA and/or siRNA target site in the 3′ or 5′ UTR of a particular mRNA, providing an accurate, specific, and highly sensitive solution to specifically blocking the binding of a particular miRNA and/or siRNA to its target site in a particular target nucleic acid without inducing degradation of the same target nucleic acid and useful in analysing miRNA:mRNA interaction and siRNA off-targeting.

### SUMMARY OF THE INVENTION

The challenges of establishing genome function and understanding the layers of information hidden in the complex transcriptomes of higher eukaryotes call for novel, improved technologies for detection and analysis of non-coding RNA and protein-coding RNA molecules in complex nucleic acid samples. The present invention solves the current problems faced by conventional approaches used in studying and modulating the interaction of mature miRNAs and/or siRNAs with their target nucleic acid(s) (e.g., mRNAs) by providing methods for the design, synthesis, and use of novel oligonucleotide compounds with improved sensitivity and high sequence specificity for RNA target sequences.

In one aspect, the present invention provides methods for analysing possible cooperative effects for miRNA and/or siRNA activity.

In another aspect the present invention features use of at least one oligonucleotide hybridising to a naturally occurring nucleotide sequence downstream or upstream of a target site of a miRNA/siRNA for identification of naturally occurring nucleotide sequences involved in regulating the activity of the miRNA/siRNA. The invention also provides a method for identifying or verifying the presence of one or more naturally occurring nucleotide sequence(s) involved in regulating the activity of a miRNA comprising a) contacting a nucleic acid sample from a subject with an oligonucleotide hybridising to a naturally occurring nucleotide sequence downstream or upstream of a target site of said miRNA, and

b) determining the activity of said miRNA in said nucleic acid sample, wherein a change in the activity identifies said naturally occurring nucleotide sequence as being involved in regulating the activity of said miRNA. In one embodiment steps a) and b) are repeated one or more times, each time using an oligonucleotide hybridising to a different or overlapping, naturally occurring nucleotide sequence downstream or upstream of the target site of the miRNA.

In preferred embodiments the at least one oligonucleotide(s) hybridising to a naturally occurring nucleotide sequence downstream or upstream of the target site of the miRNA comprises at least one high affinity nucleic acid analog. The high affinity nucleic acid analog may be LNA. In another preferred embodiment the contacting occurs in a cell. The cell sample can be of any organism in which RNA interference can occur, e.g., eukaryote, mammal, primate, human, non-human animal such as a dog, cat, horse, cow, mouse, rat, Drosophila, C. elegans, etc., plant such as rice, wheat, bean, tobacco, etc., and fungi. The cell sample can be from a diseased or healthy organism, or an organism predisposed to disease. The cell sample can be of a particular tissue type or development stage and subjected to a particular miRNA. The cell may express the miRNA endogenously or exogenously. In a further preferred embodiment of the invention the activity of the miRNA is the binding activity of the miRNA to the target site.

In preferred embodiments, the at least one oligonucleotide(s) hybridise to the naturally occurring nucleotide sequence(s) at stringent conditions.

In a further aspect the present invention provides methods to identify or verify modulators of miRNA/siRNA activity. In one embodiment, a modulator of miRNA/siRNA activity can be identified based on the nucleotide sequence of a naturally occurring nucleotide sequence downstream or upstream of a target site of a miRNA/siRNA identified as described above to be involved in regulating the activity of the miRNA/siRNA.

The present invention in a further aspect provides nucleic acid compounds comprising at least one region hybridising to a naturally occurring nucleotide sequence downstream or upstream of a target site of a miRNA, wherein the nucleic acid compound does not hybridise to the target site but is capable of inhibiting the binding of the miRNA to the target site. The at least one region is, for example, from 5-30 nucleotides, e.g., at least 10, 15, 20, or 25. The nucleic acid binds, for example, to a region located 1-500 nt, such as 10-400 nt, such as 20-300 nt, such as 30-200 nt or such as 40-100 nt, 3′ or 5′ of the miRNA/siRNA target site.

In certain embodiments such nucleic acid compounds may comprise a region hybridising at least partially to a naturally occurring nucleotide sequence identified to be involved in regulating the activity of the miRNA and/or siRNA, wherein the region may be substituted with high-affinity nucleotide analogues, e.g., LNA, to increase the sensitivity and specificity relative to conventional oligonucleotides, such as DNA or RNA oligonucleotides, for hybridization to short target sequences. The naturally occurring nucleotide sequence may be identified to be involved in regulating the activity of the miRNA and/or siRNA by the method provided above. Accordingly, in a preferred embodiment the nucleic acid composition comprises in the region the sequence of the at least one oligonucleotide hybridising to a naturally occurring nucleotide sequence downstream or upstream of a target site of a miRNA/siRNA used in the method above for identification of naturally occurring nucleotide sequences involved in regulating the activity of the miRNA/siRNA.

In one aspect, the invention provides a nucleic acid compound comprising a region hybridising to a naturally occurring nucleotide sequence including at least a portion of a site involved in the binding of at least one miRNA and/or siRNA to the target site. Alternatively, the nucleic acid compound hybridises to 100% of the site involved in regulating the binding of at least one miRNA and/or siRNA to the target site. In another embodiment, at least 10%, e.g., at least 15, 20, 25, 30, 35, 40, 45, 50, 55, 60, 65, 70, 75, 80, 85, 90, or 95%, of the nucleic acid compound is complementary to the site involved in regulating the binding of at least one miRNA and/or siRNA to the target site.

The nucleic acid is typically complementary to at least two nucleotides of the site involved in regulating miRNA and/or siRNA binding to its target site. The nucleic acid may be complementary to 3-8 nucleotides of the site involved in regulating miRNA and/or siRNA binding to its target site. For certain nucleic acid compounds the naturally occurring nucleotide sequence downstream or upstream of the miRNA target site differs by three or more nucleotides from other such sequences.

In another aspect, the invention features a method of identifying the presence of a site involved in regulating the binding of a miRNA and/or siRNA to its target site by contacting a nucleic acid sample from a subject with one or more nucleic acid compounds as described comprising a region hybridising to a naturally occurring nucleotide sequence identified to be involved in regulating the binding of a miRNA and/or siRNA and determining whether the one or more nucleic acid binds to the sample. Such methods may be employed diagnostically, as described.

In a further aspect, the invention features a method of verifying the presence of a site involved in regulating the binding of a miRNA and/or siRNA to its target site by contacting a nucleic acid sample from a subject with one or more nucleic acid compounds as described comprising a region hybridising to a naturally occurring nucleotide sequence identified to be involved in regulating the binding of a miRNA and/or siRNA and determining an expression level of a nucleic acid comprising said target site or its translation product, wherein a change in the expression level of the nucleic acid comprising the target site or its translation product verifies the presence of the site involved in the binding of a miRNA and/or siRNA to its target site.

Furthermore, the invention features a method of verifying the presence of site involved in regulating the binding of a miRNA and/or siRNA to its target site, said method comprising predicting the presence of a site involved in the binding of a miRNA and/or siRNA to its target site in a nucleic acid, such as by using a prediction algorithm, and contacting the nucleic acid sample with one or more nucleic acid compounds as described and determining an expression level of a nucleic acid comprising the target site or its translation product, wherein a change in the expression level of a nucleic acid comprising the target site or its translation product verifies the presence of the site involved in the binding of a miRNA and/or siRNA to its target site.

In further embodiments the nucleic acid compounds of the invention comprise at least one region hybridising to a naturally occurring nucleotide sequence adjacent, such as immediately adjoining or 1-2 nt, to a target site of a miRNA/siRNA, thereby inhibiting binding of the miRNA/siRNA. The nucleic acid compounds may further comprise a region non-complementary to the target site and overlapping the target site. Alternatively, the nucleic acid compounds comprise a blocking moiety, such as an alkyl chain, at the 3′- or 5′-end overlapping the target site.

In yet further embodiments the present invention is directed to nucleic acid compounds as described above comprising a first region hybridising to a naturally occurring nucleotide sequence downstream of the miRNA/siRNA target site and a second region hybridising to a naturally occurring nucleotide sequence upstream of the target site. The two regions of the nucleic acid compound may be directly connected or connected by a linker, which is non-complementary to the target site. In a preferred embodiment the linker comprises a nucleic acid sequence of between 5-20 nt, such as between 20-30 nt. In another preferred embodiment the linker comprises an alkyl chain, such as a C₁₋₁₂ alkyl chain.

In preferred embodiments, the nucleic acid compounds described herein include a high affinity nucleic acid analog, e.g. LNA. In other embodiments the nucleic acid includes a plurality of high affinity nucleotide analogs, e.g., of the same or different type. For example, the nucleic acid may include up to 80%, e.g., up to 75, 70, 65, 60, 55, 50, 45, 40, 35, 30, 25, or 20%, of the high affinity nucleic acid analog or the high affinity nucleic acid analog, e.g., LNA, in combination with one or more additional analogs, e.g., 2′-OMe. The plurality of analogs may be disposed so that no more than four naturally occurring nucleotides occur in linear sequence.

A high affinity nucleic acid analog may or may not be disposed at the 3′ or 5′ end of the nucleic acid. The nucleic acid is also preferably RNase resistant. Preferably, the nucleic acid does not prevent production of the miRNA from its corresponding pri- or pre-miRNA. In other embodiments, the analogs are not disposed in regions capable of forming auto-dimers or intramolecular complexes.

In various embodiments, a nucleic acid of the invention specifically includes one or more of 2′-O-methyl-modified nucleic acids (2′-OMe), 2′-O-(2-methoxyethyl)-modified nucleic acids (2′-MOE), 2′-Deoxy-2′-fluoro-β-D-arabinoic acid (FANA), Cyclohexene nucleic acids (CeNA), Hexitol nucleic acids (HNA) and analogs thereof, Intercalating Nucleic Acids (INA), 2′-O,4′-C-Ethylene-bridged-Nucleic Acids (ENA), and peptide nucleic acid (PNA). In other embodiments, a nucleic acid of the invention does not include 2′-O-methyl-modified nucleic acids (2′-OMe); a nucleic acid of the invention does not include 2′-O-(2-methoxyethyl)-modified nucleic acids (2′-MOE); a nucleic acid of the invention does not include 2′-Deoxy-2′-fluoro-β-D-arabinoic acid (FANA); a nucleic acid of the invention does not include Cyclohexene nucleic acids (CeNA); a nucleic acid of the invention does not include Hexitol nucleic acids (HNA) or analogs thereof; a nucleic acid of the invention does not include Intercalating Nucleic Acids (INA); a nucleic acid of the invention does not include 2′-O,4′-C-Ethylene-bridged-Nucleic Acids (ENA); and/or a nucleic acid of the invention does not include peptide nucleic acids (PNA).

The binding of the nucleic acid to the region desirably reduces the binding of the miRNA and/or siRNA to its target site, e.g., by at least 50%, 55%, 60%, 65%, 70%, 75%, 80%, 85%, or 90%. Alternatively, the nucleic acid binds to the region with a lower Kd than the miRNA and/or siRNA binds to its target site in vivo. The nucleic acid may also have an increase in binding affinity to the region as determined by an increase in Tm of at least 2° C., compared to the naturally occurring RNA complement of the region.

The invention furthermore is directed to use of one or more nucleic acid compounds of the invention for the manufacture of a medicament for treating a disease as described. The disease may or may not be caused by binding of a miRNA to a target site. The invention also features a pharmaceutical composition including one or more nucleic acids of the invention and a pharmaceutically acceptable excipient. Pharmaceutical compositions may be used in treatment of diseases associated with miRNA, as described herein. The invention also includes a diagnostic kit including one or more nucleic acids of the invention. The diagnostic kits may be employed to diagnose a disease associated with an miRNA, to prognose a subject having a disease associated with an miRNA, to determine the risk of a subject to develop a disease associated with an miRNA, or to determine the efficacy of a particular treatment for a disease associated with an miRNA. The nucleic acids of the invention may further be used as research tools and in drug screening, as described herein.

Preferred miRNAs are those associated with cancer, heart disease, cardiovascular disease, neurological diseases such as Parkinson's disease, Alzheimer's, spinal muscular atrophy and X mental retardation, atherosclerosis, postangioplasty restenosis, transplantation arteriopathy, stroke, infection, such as viral or bacterial infection, hepatitis C, psoriasis, metabolic disease, diabetes mellitus, and diabetic nephropathy.

The invention is further directed to the use of one or more nucleic acid compounds of the invention for inhibition of the binding of a miRNA to a target site and the invention further features a method of inhibiting the binding of a miRNA and/or a siRNA to its target site by contacting one or more nucleic acids of the invention with a cell expressing the target site. The contacting may occur in vitro, e.g., in drug screening, or in vivo, e.g., in therapy.

The invention also features use of one or more nucleic acid compounds of the invention for treatment of a disease, as described herein, and a method of treating a disease by contacting a subject with one or more nucleic acids of the invention in an amount sufficient to reduce binding of the miRNA to the target site, e.g., by at least 10%, 15%, 20%, 25%, 30%, 35%, 40%, 45%, 50%, 55%, 60%, 65%, 70%, 75%, 80%, 85%, 90%, or 95%. The diseases may or may not be caused by binding of an miRNA to a target site. Exemplary diseases associated with miRNA are provided herein.

The invention furthermore features a method of determining whether binding of a siRNA to a siRNA target site is associated with any unintended effects, such as off-target effects, immune response activation and/or non-specific gene silencing. In a preferred embodiment the method of determining whether a phenotype induced by a siRNA is associated with any unintended effects, such as off-target effects, immune response activation and/or non-specific gene silencing, comprises determining a phenotype of a population of cells expressing a target site for the siRNA, introducing the siRNA, and one or more nucleic acid(s) according to the present invention capable of inhibiting the binding of the miRNA to the target site, determining a phenotype of the cell population after introduction of the siRNA and one or more nucleic acid(s) according to the present invention, and comparing the two phenotypes, wherein if the phenotypes differ, the phenotype induced by the siRNA is associated with an off-target effect. In one embodiment the method of determining whether a phenotype induced by a siRNA is associated with any unintended effects, such as off-target effects, immune response activation and/or non-specific gene silencing, further comprises determining a phenotype of the population of cells after introduction of the siRNA but prior to introduction of the one or more nucleic acid(s) according to the present invention.

In preferred embodiments the determining the phenotypes in the cell populations comprises determining the expression level of a mRNA targeted by the siRNA and/or its translation product.

In preferred embodiments, the one or more nucleic acid compound(s) of the invention hybridises to the naturally occurring nucleotide sequence(s) at stringent conditions.

The nucleic acids of the invention are not splice-splice switching oligomers, e.g., of the TNFR superfamily (U.S. 2007/0105807).

### BRIEF DESCRIPTION OF THE DRAWINGS

FIG. 1 illustrates an example of a set of scanning oligonucleotides used for identifying sites in a UTR of the miRNA and/or siRNA targeted mRNA involved in regulating the activity of the miRNA and/or siRNA. Each scanning oligonucleotide hybridises to a specific sequence upstream or downstream of the miRNA and/or siRNA target site in the UTR. The scanning oligonucleotides are designed to systematically hybridize to a substantial fraction of the UTR in the vicinity of the miRNA/siRNA target site. For illustrative purposes a potential Regulatory site (site involved in regulating the activity of the miRNA/siRNA) has been included.

FIG. 2 illustrates examples of blocking oligonucleotides according to the present invention capable of inhibiting the binding of a miRNA and/or siRNA to a miRNA and/or siRNA target site. Regulatory site; site involved in regulating the activity of the miRNA/siRNA.

(A) The blocking oligonucleotide hybridises to a site upstream of the miRNA/siRNA target site involved in regulating the activity of the miRNA/siRNA.

(B) The blocking oligonucleotide hybridises to a site downstream of the miRNA/siRNA target site involved in regulating the activity of the miRNA/siRNA.

(C) The blocking oligonucleotide hybridises to a site adjacent to the miRNA/siRNA target site involved in regulating the activity of the miRNA/siRNA.

(D) The blocking oligonucleotide hybridises to a site adjacent to the miRNA/siRNA target site involved in regulating the activity of the miRNA/siRNA.

(E) The blocking oligonucleotide comprises a 5′-blocking moiety capable of interfering with the binding of the miRNA/siRNA to the miRNA/siRNA target site.

(F) The blocking oligonucleotide comprises a 3′-blocking moiety capable of interfering with the binding of the miRNA/siRNA to the miRNA/siRNA target site.

(G) The blocking oligonucleotide comprises a 5′-overhang non-complementary to the miRNA/siRNA target site capable of interfering with the binding of the miRNA/siRNA to the miRNA/siRNA target site.

(H) The blocking oligonucleotide comprises a 3′-overhang non-complementary to the miRNA/siRNA target site capable of interfering with the binding of the miRNA/siRNA to the miRNA/siRNA target site.

(I) The blocking oligonucleotide comprises a first region hybridising to a site downstream of the miRNA/siRNA target site and a second region hybridising to a site upstream of the miRNA/siRNA target site wherein the two regions are connected by a linker comprising a nucleotide sequence non-complementary to the miRNA/siRNA target site.

(J) The blocking oligonucleotide comprises a first region hybridising to a site downstream of the miRNA/siRNA target site and a second region hybridising to a site upstream of the miRNA/siRNA target site wherein the two regions are connected by a linker comprising an alkyl chain.

(K) The blocking oligonucleotide comprises a first region hybridising to a site downstream of the miRNA/siRNA target site and a second region hybridising to a site upstream of the miRNA/siRNA target site wherein the two regions are connected by a short linker comprising a nucleotide sequence non-complementary to the miRNA/siRNA target site. Binding of the blocking oligo leads to a conformational change of the target mRNA rendering it inaccessible to the miRNA/siRNA.

(L) The blocking oligonucleotide comprises a first region hybridising to a site downstream of the miRNA/siRNA target site and a second region hybridising to a site upstream of the miRNA/siRNA target site wherein the two regions are connected by a short linker comprising an alkyl chain. Binding of the blocking oligo leads to a conformational change of the target mRNA rendering it inaccessible to the miRNA/siRNA.

FIG. 3 illustrates an example of a blocking oligonucleotide according to the present invention binding to a predicted binding site of an RNA-binding protein and capable of inhibiting the binding of a miRNA and/or siRNA to a miRNA and/or siRNA target site.

### DEFINITIONS

For the purposes of the subsequent detailed description of the invention the following definitions are provided for specific terms, which are used in the disclosure of the present invention:

In the present context, the terms “blocking oligo” or “blocking molecule” refer to an oligonucleotide compound, which upon hybridisation to a target mRNA of a miRNA and/or siRNA inhibits the binding of the miRNA/siRNA to its target site.

“miRNA target site” or “microRNA target site” refer to a specific target binding sequence of a microRNA in a mRNA target. Complementarity between the miRNA and its target site need not be perfect.

Likewise, “siRNA target site” refers to a specific target binding sequence of a siRNA in a mRNA target. Complementarity between the siRNA and its target site need not be perfect.

The term “site involved in the binding of a miRNA to its target site” refers to a specific sequence of a site or region in a miRNA mRNA target, identified to be involved in the binding of a miRNA to its target site by a method such as provided herein.

Likewise, the term “site involved in the binding of a siRNA to its target site” refers to a specific sequence of a site or region in a siRNA mRNA target, identified to be involved in the binding of a siRNA to its target site by a method such as provided herein.

In the present context, the term “expression level” when refering to a nucleic acid or a translation product refers to the steady-state amount of the nucleic acid or translation product present as determined by methods known in the art and described herein.

The terms “off-target effect” or “off-targeting” in the present context refer to any gene silencing effect caused by siRNAs in non-target mRNAs through the RNAi mechanism.

In the present context “ligand” means something that binds. Ligands include biotin and functional groups such as: aromatic groups (such as benzene, pyridine, naphtalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C₁₋₂₀ alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-β-alanine, polyglycine, polylysine, peptides, oligo/polysaccharides, oligo/polyphosphates, toxins, antibiotics, cell poisons, and steroids, and also “affinity ligands”, i.e., functional groups or biomolecules that have a specific affinity for sites on particular proteins, antibodies, poly- and oligosaccharides, and other biomolecules.

The singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

“Transcriptome” refers to the complete collection of transcriptional units of the genome of any species. In addition to protein-coding mRNAs, it also represents non-coding RNAs, such as miRNAs, which have important structural and regulatory roles in the cell.

“Sample” refers to a sample of cells, or tissue or fluid isolated from an organism or organisms, including but not limited to, for example, skin, plasma, serum, spinal fluid, lymph fluid, synovial fluid, urine, tears, blood cells, organs, tumours, and also to samples of in vitro cell culture constituents (including but not limited to conditioned medium resulting from the growth of cells in cell culture medium, recombinant cells and cell components).

An “organism” refers to an entity alive at some time, including but not limited to, for example, human, mouse, rat, Drosophila, C. elegans, yeast, Arabidopsis thaliana, maize, rice, zebra fish, primates, domestic animals, etc.

The terms “detection probe” or “detection probe sequence” refer to an oligonucleotide including a recognition sequence complementary to a RNA target sequence, wherein the recognition sequence is substituted with a high-affinity nucleotide analogs, e.g., LNA, to increase the sensitivity and specificity compared to conventional oligonucleotides, such as DNA oligonucleotides, for hybridization to short target sequences, e.g., mature miRNAs, stem-loop precursor miRNAs, pri-miRNAs, as well as miRNA and/or siRNA binding sites in their cognate mRNA targets.

The terms “miRNA” and “microRNA” refer to 21-25 nt non-coding RNAs derived from endogenous genes and in the present context comprise the socalled mirtrons, produced from splicing of a short intron with hairpin potential (Berezikov et al., Mol. Cell. 28; 328-336, 2007). The miRNAs are processed from longer (ca. 75 nt) hairpin-like precursors termed pre-miRNAs. mRNAs assemble in complexes termed miRNPs and recognize their targets by antisense complementarity. If the miRNAs match 100% their target, i.e., the complementarity is complete, the target mRNA is cleaved, and the miRNA acts like a siRNA. If the match is incomplete, i.e., the complementarity is partial, then the translation of the target mRNA is blocked.

The term “siRNA” refers to 19 to 25 nt-long double-stranded small interfering RNAs. They are processed from longer double-stranded RNAs or small hairpin RNAs by the enzyme Dicer. siRNAs assemble in RISC-complexes wherein the incorporated strand acts as a guide to selectively degrade the complementary mRNA. The term “recognition sequence” refers to a nucleotide sequence that is complementary to a region within the target nucleotide sequence essential for sequence-specific hybridization between the target nucleotide sequence and the recognition sequence.

The term “label” as used herein refers to any atom or molecule which can be used to provide a detectable (preferably quantifiable) signal, and which can be attached to a nucleic acid or protein. Labels may provide signals detectable by fluorescence, radioactivity, colorimetric, X-ray diffraction or absorption, magnetism, enzymatic activity, and the like.

As used herein, the terms “nucleic acid”, “polynucleotide” and “oligonucleotide” refer to primers, probes, oligomer fragments to be detected, oligomer controls and unlabelled blocking oligomers and shall be generic to polydeoxyribonucleotides (containing 2-deoxy-D-ribose), to polyribonucleotides (containing D-ribose), and to any other type of polynucleotide which is an N glycoside of a nucleobase, e.g., purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA. The oligonucleotide is comprised of a sequence of approximately at least 3 nucleotides, preferably at least about 6 nucleotides, and more preferably at least about 8-30 nucleotides corresponding to a region of the designated target nucleotide sequence. “Corresponding” means identical to or complementary to the designated sequence. The oligonucleotide is not necessarily physically derived from any existing or natural sequence but may be generated in any manner, including chemical synthesis, DNA replication, reverse transcription or a combination thereof.

The terms “oligonucleotide” or “nucleic acid” intend a polynucleotide of genomic DNA or RNA, cDNA, semi synthetic, or synthetic origin which, by virtue of its origin or manipulation: (1) is not associated with all or a portion of the polynucleotide with which it is associated in nature; and/or (2) is linked to a polynucleotide other than that to which it is linked in nature; and/or (3) is not found in nature. Because mononucleotides are reacted to make oligonucleotides in a manner such that the 5′-phosphate of one mononucleotide pentose ring is attached to the 3′ oxygen of its neighbour in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have a 5′ and 3′ ends. When two different, non-overlapping oligonucleotides anneal to different regions of the same linear complementary nucleic acid sequence, the 3′ end of one oligonucleotide points toward the 5′ end of the other; the former may be called the “upstream” oligonucleotide and the latter the “downstream” oligonucleotide.

The linkage between two successive monomers in a nucleic acid consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—, —O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}—, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—, —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})O—, —O—P(O)₂—NR^{H}, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—, —O—P(O,S)—O—, —O—P(S)₂—O—, —NR^{H}—P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are especially desirable. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443. The left-hand side of the internucleoside linkage is bound to the 5-membered ring as substituent P* at the 3′-position, whereas the right-hand side is bound to the 5′-position of a preceding monomer.

By the term “SBC nucleobases” is meant “Selective Binding Complementary” nucleobases, i.e., modified nucleobases that can make stable hydrogen bonds to their complementary nucleobases, but are unable to make stable hydrogen bonds to other SBC nucleobases. As an example, the SBC nucleobase A′, can make a stable hydrogen bonded pair with its complementary unmodified nucleobase, T. Likewise, the SBC nucleobase T′ can make a stable hydrogen bonded pair with its complementary unmodified nucleobase, A. However, the SBC nucleobases A′ and T′ will form an unstable hydrogen bonded pair as compared to the base pairs A′-T and A-T′. Likewise, a SBC nucleobase of C is designated C′ and can make a stable hydrogen bonded pair with its complementary unmodified nucleobase G, and a SBC nucleobase of G is designated G′ and can make a stable hydrogen bonded pair with its complementary unmodified nucleobase C, yet C′ and G′ will form an unstable hydrogen bonded pair as compared to the base pairs C′-G and C-G′. A stable hydrogen bonded pair is obtained when 2 or more hydrogen bonds are formed e.g. the pair between A′ and T, A and T′, C and G′, and C′ and G. An unstable hydrogen bonded pair is obtained when 1 or no hydrogen bonds is formed e.g. the pair between A′ and T′, and C′ and G′. Especially interesting SBC nucleobases are 2,6-diaminopurine (A′, also called D) together with 2-thio-uracil (U′, also called ^{2S}U) (2-thio-4-oxo-pyrimidine) and 2-thio-thymine (T′, also called ^{2S}T) (2-thio-4-oxo-5-methyl-pyrimidine). The pairs A-^{2S}T and D-T have 2 or more than 2 hydrogen bonds whereas the D-^{2S}T pair forms a single (unstable) hydrogen bond. Likewise SBC nucleobases include pyrrolo-[2,3-d]pyrimidine-2(3H)-one (C′, also called PyrroloPyr) and hypoxanthine (G′, also called I) (6-oxo-purine), where the pairs PyrroloPyr-G and C-I have 2 hydrogen bonds each whereas the PyrroloPyr-I pair forms a single hydrogen bond.

“SBC LNA oligomer” refers to a “LNA oligomer” containing at least one LNA monomer where the nucleobase is a “SBC nucleobase”. Generally speaking SBC LNA oligomers include oligomers that besides the SBC LNA monomer(s) contain other modified or naturally occurring nucleotides or nucleosides. By “SBC monomer” is meant a non-LNA monomer with a SBC nucleobase. By “isosequential oligonucleotide” is meant an oligonucleotide with the same sequence in a Watson-Crick sense as the corresponding modified oligonucleotide e.g. the sequences agTtcATg is equal to agTscD^{2S}Ug where s is equal to the SBC DNA monomer 2-thio-t or 2-thio-u, D is equal to the SBC LNA monomer LNA-D, and ^{2S}U is equal to the SBC LNA monomer LNA ^{2S}U.

The complement of a nucleic acid sequence as used herein refers to an oligonucleotide which, when aligned with the nucleic acid sequence such that the 5′ end of one sequence is paired with the 3′ end of the other, is in “antiparallel association.” Bases not commonly found in natural nucleic acids that may be included in the nucleic acids of the present invention include, for example, inosine and 7-deazaguanine. Complementarity may not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

Stability of a nucleic acid duplex is measured by the melting temperature, or “Tₘ”. The Tₘ of a particular nucleic acid duplex under specified conditions is the temperature at which half of the duplexes have disassociated. Stability can also be used as a measure of binding affinity of an oligonucleotide towards its target.

The term “nucleobase” covers the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴, N⁴-ethanocytosin, N⁶,N⁶-ethano-2,6-diaminopurine, 5-methylcytosine, 5-(C³-C⁶)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, Nucleic Acid Research, 25: 4429-4443, 1997. The term “nucleobase” thus includes not only the known purine and pyrimidine heterocycles, but also heterocyclic analogues and tautomers thereof. Further naturally and non naturally occurring nucleobases include those disclosed in U.S. Pat. No. 3,687,808; in chapter 15 by Sanghvi, in Antisense Research and Application, Ed. S. T. Crooke and B. Lebleu, CRC Press, 1993; in Englisch, et al., Angewandte Chemie, International Edition, 30: 613-722, 1991 (see, especially pages 622 and 623, and in the Concise Encyclopedia of Polymer Science and Engineering, J. I. Kroschwitz Ed., John Wiley & Sons, pages 858-859, 1990, Cook, Anti-Cancer Drug Design 6: 585-607, 1991, each of which are hereby incorporated by reference in their entirety).

The term “nucleobase” is further intended to include heterocyclic compounds that can serve as like nucleosidic bases including certain “universal bases” that are not nucleosidic bases in the most classical sense but serve as nucleosidic bases. Especially mentioned as a universal base is 3-nitropyrrole or a 5-nitroindole. Other preferred compounds include pyrene and pyridyloxazole derivatives, pyrenyl, pyrenylmethylglycerol derivatives and the like. Other preferred universal bases include, pyrrole, diazole or triazole derivatives, including those universal bases known in the art.

By “LNA” or “LNA monomer” (e.g., an LNA nucleoside or LNA nucleotide) is meant a nucleoside or nucleotide analogue that includes at least one LNA monomer. LNA monomers as disclosed in PCT Publication WO 99/14226 are in general particularly desirable modified nucleic acids for incorporation into an oligonucleotide of the invention. Additionally, the nucleic acids may be modified at either the 3′ and/or 5′ end by any type of modification known in the art. For example, either or both ends may be capped with a protecting group, attached to a flexible linking group, attached to a reactive group to aid in attachment to the substrate surface, etc. Desirable LNA monomers and their method of synthesis also are disclosed in U.S. Pat. No. 6,043,060, U.S. Pat. No. 6,268,490, PCT Publications WO 01/07455, WO 01/00641, WO 98/39352, WO 00/56746, WO 00/56748 and WO 00/66604 as well as in the following papers: Morita et al., Bioorg. Med. Chem. Lett. 12(1):73-76, 2002; Hakansson et al., Bioorg. Med. Chem. Lett. 11 (7):935-938, 2001; Koshkin et al., J. Org. Chem. 66(25):8504-8512, 2001; Kvaerno et al., J. Org. Chem. 66(16):5498-5503, 2001; Hakansson et al., J. Org. Chem. 65(17):5161-5166, 2000; Kvaerno et al., J. Org. Chem. 65(17):5167-5176, 2000; Pfundheller et al., Nucleosides Nucleotides 18(9):2017-2030, 1999; and Kumar et al., Bioorg. Med. Chem. Lett. 8(16):2219-2222, 1998.

Preferred LNA monomers, also referred to as “oxy-LNA” are LNA monomers which include bicyclic compounds as disclosed in PCT Publication WO 03/020739 wherein the bridge between R^{4′} and R^{2′} as shown in formula (I) below together designate —CH₂—O— or —CH₂—CH₂—O—.

By “LNA modified oligonucleotide” or “LNA substituted oligonucleotide” is meant an oligonucleotide comprising at least one LNA monomer of formula (I), described infra, having the below described illustrative examples of modifications:

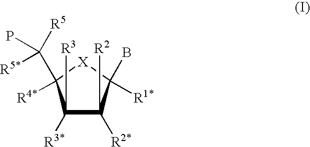

wherein X is selected from —O—, —S—, —N(R^{N})—, —C(R⁶R⁶*)—, —O—C(R⁷R⁷*)—, —C(R⁶R⁶*)—O—, —S—C(R⁷R⁷*)—, —C(R⁶R⁶*)—S—, —N(R^{N}*)—C(R⁷R⁷*), —C(R⁶R⁶*)—N(R^{N}*)—, and —C(R⁶R⁶*)—C(R⁷R⁷*).

B is selected from a modified base as discussed above e.g. an optionally substituted carbocyclic aryl such as optionally substituted pyrene or optionally substituted pyrenylmethylglycerol, or an optionally substituted heteroalicylic or optionally substituted heteroaromatic such as optionally substituted pyridyloxazole, optionally substituted pyrrole, optionally substituted diazole or optionally substituted triazole moieties; hydrogen, hydroxy, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands.

P designates the radical position for an internucleoside linkage to a succeeding monomer, or a 5′-terminal group, such internucleoside linkage or 5′-terminal group optionally including the substituent R⁵. One of the substituents R², R²*, R³, and R³* is a group P* which designates an internucleoside linkage to a preceding monomer, or a 2′/3′-terminal group. The substituents of R¹*, R⁴, R⁵, R⁵*, R⁶, R⁶*, R⁷, R⁷*, R^{N}, and the ones of R², R²*, R³, and R³* not designating P* each designates a biradical comprising about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C=Q, wherein Q is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxycarbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate optionally substituted methylene (═CH₂), and wherein two non-geminal or geminal substituents selected from R^{a}, R^{b}, and any of the substituents R¹*, R², R²*, R³, R³*, R⁴*, R⁵, R⁵*, R⁶ and R⁶*, R⁷, and R⁷* which are present and not involved in P, P* or the biradical(s) together may form an associated biradical selected from biradicals of the same kind as defined before; the pair(s) of non-geminal substituents thereby forming a mono- or bicyclic entity together with (i) the atoms to which said non-geminal substituents are bound and (ii) any intervening atoms.

Each of the substituents R¹*, R², R²*, R³, R⁴*, R⁵, R⁵*, R⁶ and R⁶*, R⁷, and R⁷* which are present and not involved in P, P* or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di-(C₁₋₆alkyl)amino, carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof.

Exemplary 5′, 3′, and/or 2′ terminal groups include —H, —OH, halo (e.g., chloro, fluoro, iodo, or bromo), optionally substituted aryl, (e.g., phenyl or benzyl), alkyl (e.g., methyl or ethyl), alkoxy (e.g., methoxy), acyl (e.g. acetyl or benzoyl), aroyl, aralkyl, hydroxy, hydroxyalkyl, alkoxy, aryloxy, aralkoxy, nitro, cyano, carboxy, alkoxycarbonyl, aryloxycarbonyl, aralkoxycarbonyl, acylamino, aroylamino, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, amidino, amino, carbamoyl, sulfamoyl, alkene, alkyne, protecting groups (e.g., silyl, 4,4′-dimethoxytrityl, monomethoxytrityl, or trityl(triphenylmethyl)), linkers (e.g., a linker containing an amine, ethylene glycol, quinone such as anthraquinone), detectable labels (e.g., radiolabels or fluorescent labels), and biotin.

In the present context, the term “C₁₋₁₂-alkyl” means a linear, cyclic or branched hydrocarbon group having 1 to 12 carbon atoms, such as methyl, ethyl, propyl, iso-propyl, cyclopropyl, butyl, tert-butyl, iso-butyl, cyclobutyl, pentyl, cyclopentyl, hexyl, cyclohexyl, and dodecyl. Analogously, the term “C₁₋₆-alkyl” means a linear, cyclic or branched hydrocarbon group having 1 to 6 carbon atoms, such as methyl, ethyl, propyl, iso-propyl, pentyl, cyclopentyl, hexyl, cyclohexyl, and the term “C₁₋₄-alkyl” is intended to cover linear, cyclic or branched hydrocarbon groups having 1 to 4 carbon atoms, e.g. methyl, ethyl, propyl, iso-propyl, cyclopropyl, butyl, iso-butyl, tert-butyl, cyclobutyl.

It is understood that references herein to a nucleic acid unit, nucleic acid residue, LNA monomer, or similar term are inclusive of both individual nucleoside units and nucleotide units and nucleoside units and nucleotide units within an oligonucleotide.

A “modified base” or other similar terms refer to a composition (e.g., a non-naturally occurring nucleobase or nucleosidic base), which can pair with a natural base (e.g., adenine, guanine, cytosine, uracil, and/or thymine) and/or can pair with a non-naturally occurring nucleobase or nucleosidic base. Desirably, the modified base provides a Tₘ differential of 15, 12, 10, 8, 6, 4, or 2° C. or less as described herein. Exemplary modified bases are described in EP 1 072 679 and WO 97/12896.

The term “chemical moiety” refers to a part of a molecule. “Modified by a chemical moiety” thus refer to a modification of the standard molecular structure by inclusion of an unusual chemical structure. The attachment of said structure can be covalent or non-covalent.

The term “inclusion of a chemical moiety” in an oligonucleotide probe thus refers to attachment of a molecular structure. Such as chemical moiety include but are not limited to covalently and/or non-covalently bound minor groove binders (MGB) and/or intercalating nucleic acids (INA) selected from a group consisting of asymmetric cyanine dyes, DAPI, SYBR Green I, SYBR Green II, SYBR Gold, PicoGreen, thiazole orange, Hoechst 33342, Ethidium Bromide, 1-O-(1-pyrenylmethyl)glycerol and Hoechst 33258. Other chemical moieties include the modified nucleobases, nucleosidic bases or LNA modified oligonucleotides.

“Oligonucleotide analog” refers to a nucleic acid binding molecule capable of recognizing a particular target nucleotide sequence. A particular oligonucleotide analogue is peptide nucleic acid (PNA) in which the sugar phosphate backbone of an oligonucleotide is replaced by a protein like backbone. In PNA, nucleobases are attached to the uncharged polyamide backbone yielding a chimeric pseudopeptide-nucleic acid structure, which is homomorphous to nucleic acid forms.

“High affinity nucleotide analogue” refers to a non-naturally occurring nucleotide analogue that increases the “binding affinity” of an oligonucleotide probe to its complementary recognition sequence when substituted with at least one such high-affinity nucleotide analogue. Commonly used analogues include 2′-O-methyl-modified nucleic acids (2′-OMe) (RNA, 2006, 12, 163-176), 2′-O-(2-methoxyethyl)-modified nucleic acids (2′-MOE) (Nucleic Acids Research, 1998, 26, 16, 3694-3699), 2′-Deoxy-2′-fluoro-β-D-arabinoic acid (FANA) (Nucleic Acids Research, 2006, 34, 2, 451-461), Cyclohexene nucleic acids (CeNA) (Nucleic Acids Research, 2001, 29, 24, 4941-4947), Hexitol nucleic acids (HNA) and analogs hereof (Nucleic Acids Research, 2001, 29, 20, 4187-4194), Intercalating Nucleic Acids (INA) (Helvetica Chimica Acta, 2003, 86, 2090-2097) and 2′-O,4′-C-Ethylene-bridged-Nucleic Acids (ENA) (Bioorganic and Medicinal Chemistry Letters, 2002, 12, 1, 73-76). Additionally, in the present context, the oligonucleotide mimic referred to as peptide nucleic acid (PNA) (Nielsen et al., Science 254; 1497-1500, 1991 and U.S. Pat. Nos. 5,539,082; 5,714,331; and 5,719,262) is considered a high affinity nucleotide analogue. A preferred high affinity nucleotide analogue is LNA. A plurality of a combination of analogues may also be employed in an oligo of the invention.

As used herein, an oligo with an increased “binding affinity” for a recognition sequence compared to an oligo that includes the same sequence but does not include a nucleotide analog, refers to an oligo for which the association constant (Kₐ) of the recognition segment is higher than the association constant of the complementary strands of a doublestranded molecule. In a preferred embodiment, the association constant of the recognition segment is higher than the dissociation constant (K_{d}) of the complementary strand of the recognition sequence in the target sequence in a double stranded molecule.

Monomers are referred to as being “complementary” if they contain nucleobases that can form hydrogen bonds according to Watson-Crick base-pairing rules (e.g. G with C, A with T or A with U) or other hydrogen bonding motifs such as for example diaminopurine with T, 5-methyl C with G, 2-thiothymidine with A, inosine with C, pseudoisocytosine with G, etc.

The term “succeeding monomer” relates to the neighbouring monomer in the 5′-terminal direction and the “preceding monomer” relates to the neighbouring monomer in the 3′-terminal direction.

The term “target nucleic acid” or “target ribonucleic acid” refers to any relevant nucleic acid of a single specific sequence, e.g., a biological nucleic acid, e.g., derived from a patient, an animal (a human or non-human animal), a plant, a bacteria, a fungi, an archae, a cell, a tissue, an organism, etc. For example, where the target ribonucleic acid or nucleic acid is derived from a bacteria, archae, plant, non-human animal, cell, fungi, or non-human organism, the method optionally further comprises selecting the bacteria, archae, plant, non-human animal, cell, fungi, or non-human organism based upon detection of the target nucleic acid. In one embodiment, the target nucleic acid is derived from a patient, e.g., a human patient. In this embodiment, the invention optionally further includes selecting a treatment, diagnosing a disease, or diagnosing a genetic predisposition to a disease, based upon detection of the target nucleic acid.

“Target sequence” refers to a specific nucleic acid sequence within any target nucleic acid.

The term “stringent conditions”, as used herein, is the “stringency” of hybridisation which occurs within a range from about Tₘ−5° C. (5° C. below the melting temperature (Tₘ) of the probe) to about 20° C. to 25° C. below Tₘ. As will be understood by those skilled in the art, the stringency of hybridization may be altered in order to identify or detect identical or related polynucleotide sequences. Hybridization techniques are generally described in Nucleic Acid Hybridization, A Practical Approach, Ed. Hames, B. D. and Higgins, S. J., IRL Press, 1985; Gall and Pardue, Proc. Natl. Acad. Sci., USA 63: 378-383, 1969; and John, et al. Nature 223: 582-587, 1969.

### DETAILED DESCRIPTION OF THE INVENTION

mRNAs and siRNAs target mRNA sequences in a sequence specific manner by inducing mRNA degradation or inhibiting protein synthesis by blocking translation. It has recently been demonstrated that each miRNA may have multiple targets, in some cases up to several hundreds and off target gene silencing mediated by either strand of siRNAs designed according to presently available design rules and algorithms can result in undesired changes in the expression of several genes and induce measurable phenotypes.

To study these effects of miRNAs and siRNAs one option is to introduce an oligonucleotide complementary to the miRNA or siRNA guide strand, in this way blocking the effect of the miRNA or siRNA. However, this will either potentially induce or block the effect on multiple genes, that may be targeted by the miRNA or siRNA.

It is therefore desirable to have available reagents that can specifically block the effect of individual miRNAs/siRNAs or antisense molecules on single genes, rather than addressing multiple genes simultaneously. One way to achieve this is by transfecting cells with nucleic acid constructs that are homologous to the target site sequence of the miRNA/siRNA and are able to block this site thereby making it inaccessible to the regulatory miRNA/siRNA.

When designing nucleic acid constructs that are homologous to the target sequence of the miRNA and are able to block this site by making it inaccessible to the miRNA, it is important to acknowledge, that a large part of the miRNA target sequence is conserved among multiple targets in the transcriptome. Recent work from Brennecke et al. (PloS Biol 3(3): e85, 2005) has provided evidence that an average miRNA has approximately 100 target sites, indicating that miRNAs regulate a large fraction of protein-coding genes. Hence, if the blocking oligonucleotide is targeted against the target site sequence of the specific gene transcript, it may or may not also block target sequences at other genes, which may not be desirable.

One way to overcome this problem may be to design blocking oligonucleotides which bind to the miRNA target site as well as to a gene-specific region adjacent to the miRNA target site. The PCT patent application No. PCT/DK2007/000565 describes miRNA target site blocking oligos binding to a region comprising a portion of a miRNA target site and a naturally occurring nucleic acid sequence adjacent to said miRNA target site.

### Design Parameters

Another way to overcome this problem is to design blocking oligonucleotide compounds according to the present invention which hybridise to transcript-specific sequences which do not comprise portions of the miRNA/siRNA target site. These blocking oligonucleotides may be capable of interfering with miRNA/siRNA binding physically by rendering the miRNA/siRNA target site inaccessible to the miRNA/siRNA or indirectly by blocking a sequence involved in regulating the binding of a miRNA/siRNA, such as a sequence recognized by i.e. a modulator or cofactor of miRNA/siRNA activity, a protein of the miRNP/RISC complex or another miRNA acting as a co-factor.

A method for identification of target mRNA regions affecting on the activity of a specific miRNA/siRNA may include developing a model system for testing possible cooperative effects in miRNA/siRNA binding. One example of such a model system is a cell line wherein the effect of a specific miRNA/siRNA on a particular mRNA target is well-characterized. Another example is a cell line expressing the specific miRNA/siRNA and a reporter construct comprising a 3′ or 5′ UTR of the particular mRNA target with a verified target site for the miRNA/siRNA and a reporter gene, such as luciferase (luc). Additionally, comparison of several cell lines expressing the same miRNA/siRNA and mRNA target pair may also lead to identification of possible combinations of additional binding site and possible cofactors of miRNA/siRNA activity against the target. In preferred embodiments the analyzed mRNA target contains multiple miRNA target sites and/or binding sites for other miRNAs/siRNAs.

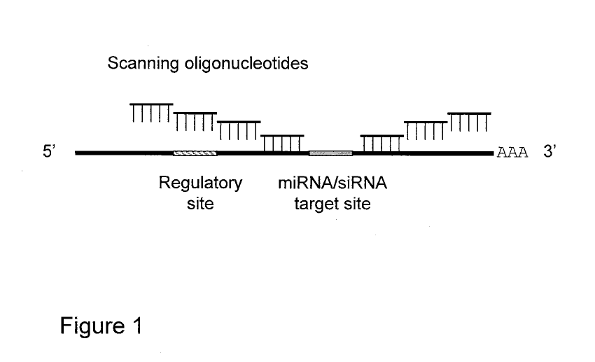

One or more oligonucleotides may be synthesized and transfected one-by-one into identical samples of a cell line expressing a specific miRNA/siRNA, by methods known in the art, to hybridise to specific regions of the mRNA. In a preferred embodiment, several such “scanning” oligonucleotides with overlapping or flanking recognition sequences (see FIG. 1 for design of a set of “scanning” oligonucleotides) are synthesized and transfected one-by-one into the cells to systematically cover substantial regions downstream and upstream of the miRNA/siRNA target site. The effect of each oligonucleotide on miRNA/siRNA activity is determined by comparing the level of miRNA/siRNA activity in a sample of cells not transfected with an oligonucleotide to the level of miRNA/siRNA activity in a transfected sample of cells, f.ex. by determining expression levels of target mRNA and/or its translation product or the expression levels of a reporter gene as described herein. In a preferred embodiment, the effect on miRNA/siRNA activity is determined by comparing the levels of miRNA/siRNA:mRNA interaction. In one embodiment the specific sequence of the mRNA, which is targeted by an oligonucleotide, is identified as being involved in regulating the binding of the particular miRNA/siRNA to the miRNA/siRNA target site in the mRNA if transfection of said oligonucleotide results in a change in the activity of the miRNA/siRNA. In another embodiment, the specific sequence of the mRNA, which is targeted by an oligonucleotide, is identified as being involved in regulating the binding of the particular miRNA/siRNA to the miRNA/siRNA target site in the mRNA if transfection of said oligonucleotide results in a reduction of miRNA/siRNA:mRNA interaction by at least 50%, such as at least 60%, such as at least 70%, such as at least 80%, such as at least 90%.

When a sequence has been identified to be involved in regulating the binding of a miRNA and/or siRNA, cells can be transfected with an oligonucleotide compound which hybridises to at least a portion of the identified sequence and thereby inhibit miRNA/siRNA activity on the specific targeted mRNA. The identified sites may be found to be conserved among multiple targets in the genome. Thus in certain embodiments the oligonucleotide compound hybridises to at least a portion of the identified sequence as well as to a gene-specific region adjacent to the identified site. In one embodiment the oligonucleotide compound is similar or identical to the oligonucleotide used to identify the site as a site involved in regulating miRNA activity. Other oligonucleotide compounds which hybridise to a naturally occurring nucleotide sequence upstream and downstream of a miRNA and/or siRNA target site in the 3′ or 5′ UTR of a particular mRNA are described herein.

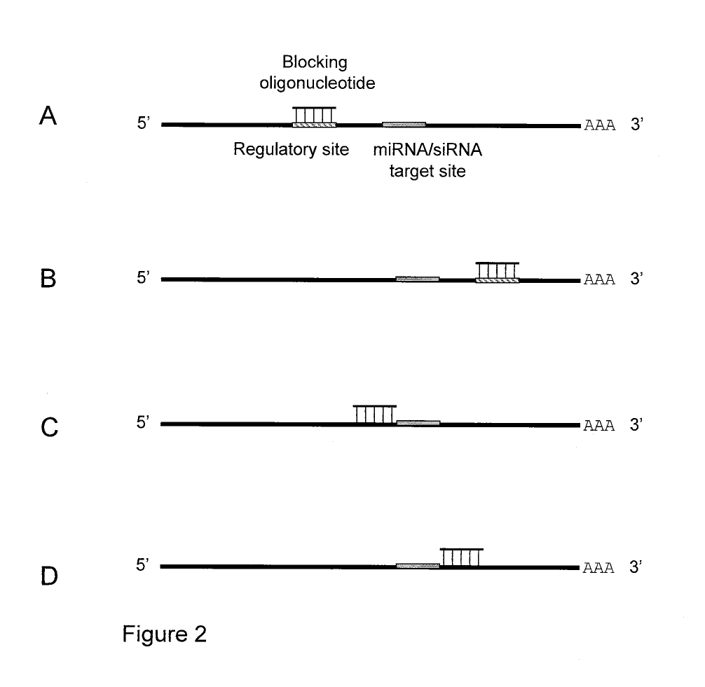

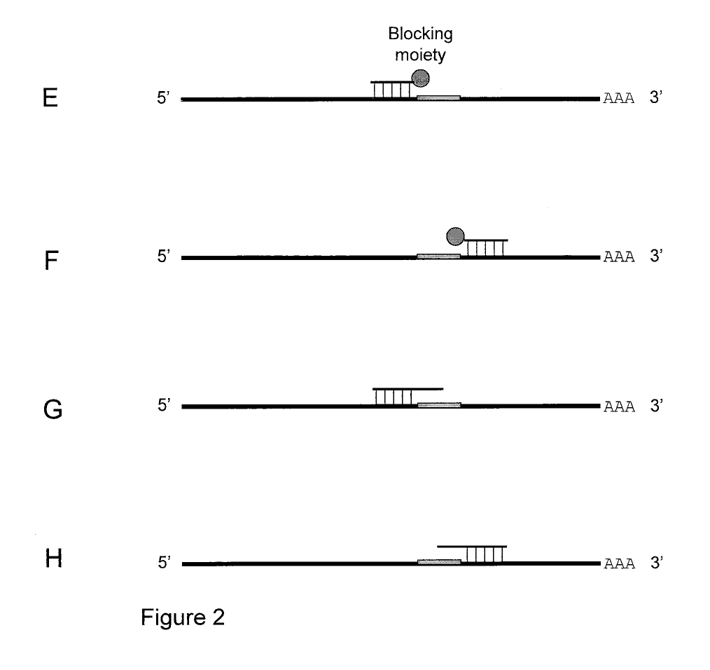

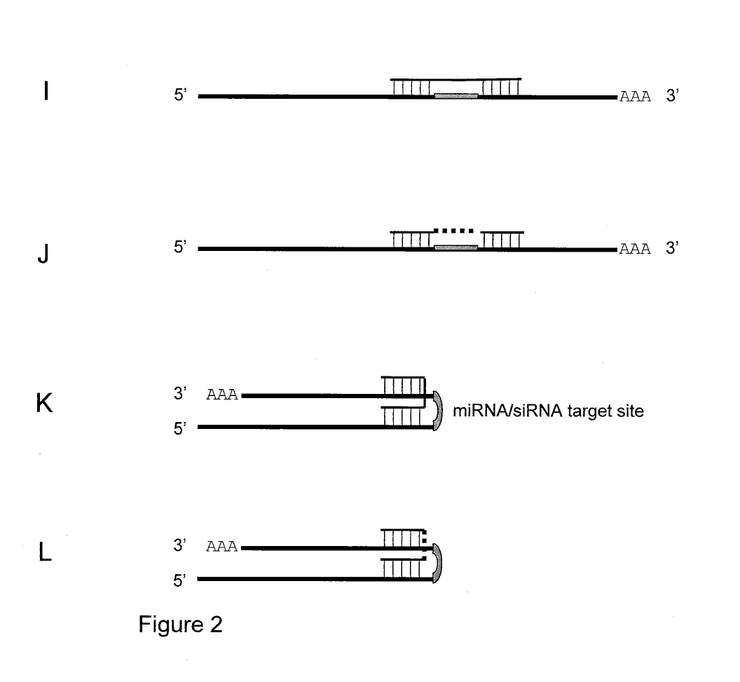

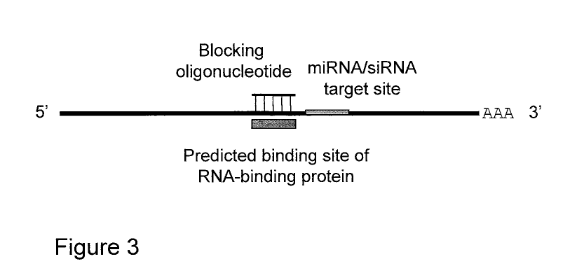

One advantageous principle of designing gene-specific miRNA/siRNA blocking oligos is to design an oligonucleotide compound sterically interfering with miRNA/siRNA binding to the miRNA/siRNA target site (FIG. 2, C-L). Assuming that miRNA/siRNA binding is assisted by proteins, such as in a miRNP/RISC complex) the area on the target sequence required for binding of a miRNA/siRNA to a target mRNA sequence could be wider than the length of the miRNA/siRNA itself. It could therefore be proposed that binding of a miRNA/siRNA to a target mRNA sequence could be inhibited by applying oligonucleotides that bind adjacent, such as immediately adjoining or 1-2 nt, to the target site but not overlapping the target site. Thus, in certain embodiments, the oligonucleotide compound is designed to hybridise to a sequence adjacent, such as immediately adjoining or 1-2 nt, to the miRNA/siRNA target site (FIG. 2, C-D).

Alternatively, the blocking oligos could comprise a non-complementary sequence overhang or other moiety that overlap the target site. Therefore, in other embodiments, the oligonucleotide compound may comprise a non-complementary nucleic acid sequence (FIG. 2, G-H) or a blocking moiety, such as f.ex an alkyl chain (FIG. 2, E-F), partly overhanging the miRNA/siRNA target site upon hybridisation. In further embodiments, the oligonucleotide compound comprises two recognition regions. The first region is 5-25 nt long and designed to bind to a sequence 1-5 nt downstream of the miRNA/siRNA target site and the second region is 5-25 nt long and designed to bind to a sequence 1-5 nt upstream of the miRNA/siRNA target site. The two regions are connected by a 10-30 nt long linker not complimentary to the miRNA/siRNA target site. In one embodiment the linker comprises a stretch of DNA or RNA monomers (FIG. 2, I). In other embodiments the linker comprises a C₁₋₁₂ alkyl chain (FIG. 2, J). Two target-specific regions will ensure additional specificity and affinity for target mRNA recognition and blocking.

Another advantageous principle is to use an oligonucleotide compound designed to change the conformation of a region comprising the miRNA/siRNA target site. One example is an oligonucleotide compound comprising two recognition regions. The first region is 5-25 nt long and designed to bind to a sequence 1-500 nt downstream of the miRNA/siRNA target site and the second region is 5-25 nt long and designed to bind to a sequence 1-500 nt upstream of the miRNA/siRNA target site. The two regions may or may not be connected by a short (1-5 nt) stretch of DNA or RNA monomers or a short C₁₋₄ alkyl chain and the binding of the two regions to the target mRNA will create an internal loop which include the miRNA/siRNA target site (FIG. 1, K-L).

It is of utmost importance that the blocking molecule can hybridise with high affinity compared to miRNA siRNA, that it has high nuclease resistance, and very importantly that it does not induce antisense (e.g., RNAseH induction) effect on the target molecule. High affinity nucleic acid analog (e.g., LNA) containing molecules have several advantages for this purpose:
Nucleic acid analogs can increase thermal stability allowing the blocking molecule to bind preferentially to a gene-specific sequence downstream and/or upstream from the target site.
Nucleic acid analog containing molecules show increased stability compared to normal nucleic acid molecules either DNA or RNA
It has been shown that when employing nucleotide analogues such as LNAs for antisense molecules, a 5-8 nucleotide centrally located “gap” of un-modified DNA or RNA molecules is necessary to induce RNAseH mediated degradation of the target (Kurreck et al., Nucleic Acids Res. 30(9); 1911-1918, 2002).
It has recently been shown that the positioning of nucleic acid analogs, such as LNA, at specific locations, e.g. in the 5′ and/or 3′ ends, of siRNA molecules can inhibit the siRNA-mediated decay of the target messenger.
Furthermore it has been demonstrated that LNA does not induce interferon response in in vivo administration.

Thus, nucleic acid molecules, which do not induce antisense (e.g., RNAseH induction) effects on the target molecule can be designed to block gene-specific target site binding of miRNA or siRNA.

Another advantageous design principle would be to include, at least, inter-spaced nucleic acid analogues in the entire sequence, to prevent the formation of gapmers and RNase H-mediated degradation. Gaps should not exceed 4 nucleotides.

Yet another advantageous design feature would be to include in the blocking molecule nucleic acid analogues in the 3′ and 5′ ends to enhance bio-stability and to decrease liability to intracellular nucleases and siRNA-mediated decay.

Determining and Modulating the Functional Role of miRNAs

Prediction software often bases predictions of miRNA target sites on perfect complementarity of target 5′ seed sequences and partial complementarity of the remaining sequences. Since miRNAs often elicit their effect through incomplete binding to target nucleotide sequences, bioinformatically predicting the target nucleotides (e.g., mRNAs) of a given miRNA based on its sequence alone is not trivial and may not provide evidence that an interaction is occurring in vivo. One way of experimentally investigating the interaction between a miRNA and its target is to inactivate the miRNA in question (e.g., by providing a complementary knock-down oligo). However, each miRNA may have multiple target nucleic acids (e.g., mRNAs) in the cell, in some case more than 200 predicted targets may exist for a given miRNA. Hence, inactivating a specific miRNA in a cell may not directly provide evidence for interaction between a specific miRNA and a target nucleic acid (e.g., mRNA), since potential effects may be elicited by interactions between the miRNA and other targets in the cell.

A challenge in miRNA research is therefore to establish evidence that an interaction occurs between a miRNA and a prediction miRNA target site in a target nucleic acid (e.g., mRNA). By providing a method by which to specifically block binding of a miRNA to a particular miRNA target site in a particular target nucleotide, the present invention provides a solution to study the specific interaction between a miRNA and its target.

Exemplary miRNAs and their targets are described herein and are known in the art, e.g. in the miRBase Sequence Database (D140-D144 Nucleic Acids Research, 2006, Vol. 34, Database Issue) and miRGen (D149-D155 Nucleic Acids Research, 2006, Vol. 35, Database Issue), each of which is hereby incorporated by reference.

Modulating miRNA Interactions for Specific Target Nucleotides.

MiRNAs have been shown to be involved in several types of diseases, as described herein, and therapeutic strategies have been contemplated where specific miRNAs are blocked or inhibited, to treat miRNA related diseases. However, as described, each miRNA may have multiple target nucleic acids (e.g., mRNAs) in the cell. Hence, inactivating a specific miRNA in a cell will not only affect the interaction between a specific miRNA and a target nucleic acid (e.g., mRNA) but will also affect interactions between the miRNA and other targets in the cell generally. In strategies to develop therapeutic agents to treat miRNA mediated diseases, a challenge in miRNA research has been therefore to establish a method to modulate an interaction that occurs between a specific miRNA and one or more specific miRNA target sites in one or more specific target nucleic acids (e.g., mRNA). By providing a method by which to specifically block the binding of a particular miRNA to a specific target site in a particular target nucleic acid, the present invention provides a solution to develop specific therapeutic agents directed against miRNA mediated diseases

Identification and Validation of miRNA Target Sites

In order to design miRNA blocking oligonucleotides according to the present invention, functional miRNA sites need to be identified and preferably validated.

Computational predictions have been the mainstay for discovery of miRNAs and their targets. Multiple computer prediction algorithms have been developed that use established miRNA-mRNA interaction rules, some of which have been trained on existing microarray data, to identify miRNA targets (John et al., PloS Biol 2; e363, 2004; Krek et al., Nature Genetics 37; 495-500, 2005; Kiriakidou et al., Genes Dev 18; 1165-1178; Lewis et al., Cell 120; 15-20, 2005; Robins and Padgett, PNAS 102; 4006-4009, 2005). The algorithms used to predict miRNa targets typically develop scoring schemes based on sequence complementarity, free energy calculations of RNA duplex formation, phylogenetic conservation, and target RNA structure. Several algorithms are readily available on the web, such as TargetScan at the MIT website (Lewis et al., Cell 120; 15-20, 2005), Miranda at the microRNA website (John et al., PloS Biol 2; e363, 2004), PicTar at the New York University website (Krek et al., Nature Genetics 37; 495-500, 2005) and DIANA-microT at the University of Pennsylvania website (Kiriakidou et al., Genes Dev 18; 1165-1178).

One method for identification of miRNA targets relies on measuring reductions in the amounts of target mRNA caused by an exogenously added miRNA (Lim et al., Nature 433; 769-773, 2005).

Recently, direct biochemical methods that combine RNA-induced silencing complex (RISC) purification with microarray analysis bound mRNAs have been used for miRNA target discovery (Karginov et al., PNAS104(49); 19291-19296; Easow et al., RNA 13(8); 1198-1204, 2007).

US2004/0175732 describes further methods of identifying miRNA targets, whether or not the sequence of the miRNA is known, by obtaining an miRNA/target RNA complex and transcribe target complementary RNA from the target RNA. cDNA is synthesized and the cDNA is sequenced.

Potential targets can typically be validated by using luciferase reporters containing the target 3′UTR.

Blocking Oligo Desirably Blocks miRNA/siRNA Target Binding Efficiently.

To determine the effect of miRNA or siRNA blocking oligonucleotides, a relative measure comparing the range between 1) the expression level of a given miRNA or siRNA target nucleotide or resulting protein under an approximate maximum effect of a miRNA (e.g., given the natural miRNA level in a specific cell or the effect of over expression of the miRNA) and 2) the expression level of the miRNA or siRNA target nucleotide or resulting protein without the miRNA or siRNA present (e.g., in a cell not expressing the miRNA or siRNA or by co-transfecting with a miRNA or siRNA knockdown probe) with 3) the expression level of the miRNA target nucleotide or resulting protein under an approximate maximum effect of a miRNA or siRNA (e.g., over expression of the miRNA or siRNA) and in the presence of a given concentration of a miRNA or siRNA blocking oligo targeting the same miRNA or siRNA target nucleotide may be determined. For example, if the amount of the miRNA or siRNA target mRNA or resulting protein is changed by 50% of the range between 1) and 2) by addition of the miRNA or siRNA blocking oligo of a given concentration under approximate maximum effect of the miRNA or siRNA, the target site blocking oligo will have blocked 50% of the miRNA or siRNA effect at that given concentration.

The amount of the target may be reflected in the relative expression level of the miRNA or siRNA target nucleotide (e.g., a messenger RNA as determined by QPCR or northern blot or similar technologies known in the art) or in the relative expression level of the translational product (protein, as determined by e.g. Western blotting or by measuring the enzymatic or catalytic activity of the resulting protein (e.g. as lucifierase actitivty in case of the luciferase enzyme)) of the miRNA or siRNA target mRNA.

### Methods for Determining Biological State and Biological Response.

This invention provides methods comprising determining response profiles, of specific blocking of miRNA activity. The measured responses can be measurements of cellular constituents in a cell or organism or responses of a cell or organism to a specific blocking of miRNA activity. The cell sample can be of any organism in which RNA interference can occur, e.g., eukaryote, mammal, primate, human, non-human animal such as a dog, cat, horse, cow, mouse, rat, Drosophila, C. elegans, etc., plant such as rice, wheat, bean, tobacco, etc., and fungi. The cell sample can be from a diseased or healthy organism, or an organism predisposed to disease. The cell sample can be of a particular tissue type or development stage and subjected to a particular miRNA. One of skill in the art would appreciate that this invention is not limited to the following specific methods for measuring the expression profiles and responses of a biological system.

### Methods of Transcriptional State Measurement.

The transcriptional state of a cell may be measured by gene expression technologies known in the art. Several such technologies produce pools of restriction fragments of limited complexity for electrophoretic analysis, such as methods combining double restriction enzyme digestion with phasing primers (see, e.g., European Patent O 534858 A1, filed Sep. 24, 1992, by Zabeau et al.), or methods selecting restriction fragments with sites closest to a defined mRNA end (see, e.g., Prashar et al., 1996, Proc. Natl. Acad. Sci. USA 93:659-663). Other methods statistically sample cDNA pools, such as by sequencing sufficient bases (e.g., 20-50 bases) in each of multiple cDNAs to identify each cDNA, or by sequencing short tags (e.g., 9-10 bases) that are generated at known positions relative to a defined mRNA end (see, e.g., Velculescu, 1995, Science 270:484-487).

The expression level of a nucleotide sequence in a gene can be measured by any high throughput techniques. However measured, the result is either the absolute or relative amounts of transcripts or response data, including but not limited to values representing abundance ratios. Preferably, measurement of the expression profile is made by hybridization to transcript arrays, such as described in PCT patent application no. WO 2005/18534.

The relative abundance of an mRNA and/or an exon expressed in an mRNA in two cells or cell lines is scored as different (i.e., the abundance is different in the two sources of mRNA tested) or as identical (i.e., the relative abundance is the same). As used herein, a difference between the two sources of RNA of at least a factor of about 25% (i.e., RNA is 25% more abundant in one source than in the other source), more usually about 50%, even more often by a factor of about 2 (i.e., twice as abundant), 3 (three times as abundant), or 5 (five times as abundant) is scored as different. Present detection methods allow reliable detection of difference of an order of about 3-fold to about 5-fold, but more sensitive methods are expected to be developed.

### Embodiments Based on Translational State Measurements

Gene expression data may include translational state measurements or even protein expression measurements. Measurement of the translational state may be performed according to several methods. Proteins can be separated and measured by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of proteins: A Practical Approach, IRL Press, New York; Shevchenko et al., 1996, Proc. Natl. Acad. Sci. USA 93:1440-1445; Sagliocco et al., 1996, Yeast 12:1519-1533; Lander, 1996, Science 274:536-539; and Beaumont et al., Life Science News 7, 2001, Amersham Pharmacia Biotech. The resulting electropherograms can be analyzed by numerous techniques, including mass spectrometric techniques, Western blotting and immunoblot analysis using polyclonal and monoclonal antibodies, and internal and N-terminal microsequencing. Using these techniques, it is possible to identify a substantial fraction of all the proteins produced under given physiological conditions, including in cells (e.g., in yeast) exposed to a miRNA and/or a blocking oligo of the invention, or in cells modified by, e.g., deletion or over-expression of a specific gene.

In some embodiments, rather than using gene expression interaction maps based on gene expression, protein expression interaction maps based on protein expression maps are used. For example, whole genome monitoring of protein (i.e., the “proteome,” Goffeau et al., 1996, Science 274:546-567; Aebersold et al., 1999, Nature Biotechnology 10:994-999) can be carried out by constructing a microarray in which binding sites comprise immobilized, preferably monoclonal, antibodies specific to a plurality of protein species encoded by the cell genome (see, e.g., Zhu et al., 2001, Science 293:2101-2105; MacBeath et al., 2000, Science 289:1760-63; de Wildt et al., 2000, Nature Biotechnology 18:989-994). Preferably, antibodies are present for a substantial fraction of the encoded proteins, or at least for those proteins relevant to the action of a miRNA of interest. Methods for making monoclonal anti-bodies are well known (see, e.g., Harlow and Lane, 1988, Antibodies: A Laboratory Manual, Cold Spring Harbor, N.Y., which is incorporated in its entirety for all purposes). In a preferred embodiment, monoclonal antibodies are raised against synthetic peptide fragments designed based on genomic sequence of the cell. With such an antibody array, proteins from the cell are contacted to the array and their binding is assayed with assays known in the art.

### Embodiments Based on Other Aspects of the Biological State

The methods of the invention are applicable to any cellular constituent that can be monitored. In particular, where activities of proteins can be measured, embodiments of this invention can use such measurements. Activity measurements can be performed by any functional, biochemical, or physical means appropriate to the particular activity being characterized. Where the activity involves a chemical transformation, the cellular protein can be contacted with the natural substrate(s), and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In alternative and non-limiting embodiments, response data may be formed of mixed aspects of the biological state of a cell. Response data can be constructed from, e.g., changes in certain mRNA abundances, changes in certain protein abundances, and changes in certain protein activities.

Blocking Oligo Desirably Only Blocks Binding to a Single miRNA or siRNA Target Site, Specific to a Particular mRNA.

It is preferable that the blocking oligo can be designed to target only a single specific mRNA. Since each miRNA (or siRNA) may target multiple mRNAs, target sites as well as sites involved in regulating binding of the miRNA/siRNA to the target site in different mRNAs may be very similar, hence allowing a blocking oligo designed to inhibit binding to one specific target site, to also block other target sites. This can be avoided by designing the blocking oligo to cover an mRNA sequence adjacent to the target site or site involved in regulating binding to the target site, since this sequence will likely be specific for the mRNA in question. Furthermore, the oligo designed can be compared to a database comprising the complete transcriptome (e.g., by a BLAST search). The oligo sequence preferably should not occur more than once in such a database. Preferably, similarity with other sites differing by fewer than 2 nucleotides in identity is avoided.

Blocking Oligo Desirably does not Target pri- or pre-miRNA or Precursor siRNA Molecules to Avoid Blocking of Endogenous miRNA/siRNA Production.

The sequence of the blocking oligo will be at least partially identical to the targeting miRNA/siRNA sequences. Since miRNAs are produced from processed pri- and pre-miRNA molecules comprising hairpins structures involving the sequence of the mature miRNA and siRNAs may be produced from longer double stranded RNA or small hairpin RNA, a miRNA/siRNA target site blocking oligo comprising the complete miRNA/siRNA target sequence might function to block the pre-mir and/or precursor siRNA hence eliminating the production of the specific miRNA/siRNA.

Use of Several Blocking Oligos may be Required to Protect a Specific mRNA from Degradation.

One miRNA can have binding sites in multiple target nucleic acids and one target sequence can be targeted by multiple miRNAs. Moreover, several binding sites for one miRNA can be found in the 3′UTR of an mRNA.

Accessible siRNA target sites may be rare in some mammalian mRNAs. However, more effective gene silencing can be achieved by targeting different segments of the same transcript simultaneously with two or more siRNAs against different sites of the same mRNA (Ji et al., FEBS Lett. 552; 247-252, 2003).

Thus, in certain embodiments the present invention provides for the administration of more than one nucleic acid compounds as described herein for blocking of more than one miRNA and/or siRNA target sites of a particular target mRNA.

In one embodiment the present invention provides for the treatment of a patient with a miRNA associated disease by administering to said patient more than one oligonucleotide as described herein for blocking of more than one miRNA target sites.

### Resistance to Degradation

It has been shown (Kurreck et al., Nucleic Acids Research 30(9); 1911-1918, 2002) that LNA/DNA mixmers do not induce significant RNase H cleavage. A gap in a chimeric LNA/DNA oligonucletoide is needed to recruit RNase H and a DNA stretch of 7-8 nucleotides was found to provide full activation of RNase H. For gap mers with 2′-O-methyl modifications a shorter stretch of only six deoxy monomers is sufficient to induce efficient RNase H cleavage.

In a preferred embodiment, the single stranded oligonucleotide according to the invention does not mediate RNase H based cleavage of a complementary single stranded RNA molecule.

EP 1 222 309 provides in vitro methods for determining RNase H activity, which may be used to determine the ability to recruit RNase H. A compound is deemed essentially incapable of recruiting RNAse H if, when provided with the complementary RNA target, and RNase H, the RNase H initial rate, as measured in pmol/l/min, is less than 20%, such as less than 10%, such as less than 5%, or less than 1% of the initial rate determined using the equivalent DNA only oligonucleotide using the methodology provided by Example 91-95 of EP 1 222 309.

A compound is deemed capable of recruiting RNase H if, when provided with the complementary RNA target it has an initial rate, as measured in pmol/l/min, of at least 1% such as at least 5%, such as at least 10% or less than 20% of the equivalent DNA only oligonucleotide using the methodology provided by Example 91-95 of EP 1 222 309.

miRNA and Disease

miRNAs that are associated with disease either through their upregulation or downregulation of transcripts may be used for diagnostic or therapeutic targets. For example, when expression of an miRNA results in downregulation or upregulation of a transcript associated with a disease, administration of an amount of a blocking nucleic acid of the invention sufficient to reduce the in vivo produces a therapeutic effect. Alternatively, a nucleic acid of the invention may be used in a diagnostic to determine whether the target is present in a sample from a subject, e.g., to determine risk for a disease caused by a miRNA binding to the target site or to determine suitability of a particular therapeutic. When underexpression of an miRNA or its target sequence is associated with a disease, nucleic acids of the invention may be used as diagnostics, as described herein. Exemplary diseases that are associated with miRNAs include cancer, heart disease, cardiovascular disease, neurological diseases such as Parkinson's disease, Alzheimer's, spinal muscular atrophy and X mental retardation, atherosclerosis, postangioplasty restenosis, transplantation arteriopathy, stroke, infection, such as viral or bacterial infection, hepatitis C, psoriasis, metabolic disease, diabetes mellitus, and diabetic nephropathy. Specific miRNAs and diseases are further described below.

miRNAs are reported to be associated with the pathogenesis of a large range of human diseases (Soifer et al., Molecular Therapy 15(12); 2070-2079, 2007 for review). miRNA expression profiles demonstrate that many miRNAs are deregulated in human cancers. miRNAs have been shown to regulate oncogenes, tumor suppressors and a number of cancer-related genes controlling cell cycle, apoptosis, cell migration and angiogenesis. miRNAs encoded by the mir-17-92 cluster have oncogenic potential and others may act as tumor suppressors. Some miRNAs and their target sites have been found to be mutated in cancer (Calin and Croce, Nature Reviews 6; 857-866, 2006; Esquela-Kerscher and Slack, Nature Reviews 6; 259-269, 2006 for reviews)

Other specific miRNAs are misexpressed in diseased hearts, and gain- and loss-of-function experiments in mice have shown these miRNAs to be necessary and sufficient for multiple forms of heart disease (Rooij and Olsen, J Clin Invest 117: 2369-2376, 2007 for review).

Recent studies (Martin et al., JBC 282(33); 24262-24269, 2007) demonstrate that miR-155 translationally represses the expression of Angiotensin II type 1 receptor (AT1R) in vivo. A silent polymorphism in the human AT1R gene has been associated with cardiovascular disease.

MiRNAs have recently been implicated in the intricate cross-talk between the host and pathogen in viral infections and is thought to play a major role in viral pathogenesis (Scaria et al., Retrovirology 3; 68, 2006 for review).

Recently, it has been shown that miRNA are active during embryogenesis of the mouse epithelium and play a significant role in skin morphogenesis (Yi et al., Nature Genetics 38(3); 273-274, 2006) and a strong indication for the involvement of miR-203 in the pathogenesis of psoriasis has been presented (Sonkoly et al., PLoS ONE. 2(7):e610, 2007).

The abundant expression of miRNAs in the brain highlights their biological significance in neurodevelopment. It was recently shown that miR-124 directly targets PTBPI (PTB/hnRNP I) mRNA, which encodes a global repressor of alternative pre-mRNA splicing in nonneuronal cells and promotes the development of the nervous system (NS), at least in part by regulating an intricate network of NS-specific alternative splicing (Makeyev et al., Molecular Cell 27(3); 435-448, 2007 Dec. 11).

Target prediction and in vitro functional studies have shown that MITF, a transcription factor required for the establishment and maintenance of retinal pigmented epithelium, is a direct target of miR-96 and miR-182 (Xu et al., JBC 282(34); 25053-25066, 2007).

Additionally miRNAs have been shown to play a role in insulin secretion and glucose homeostasis. Overexpression of miR-375 suppressed glucose-induced insulin secretion and inhibition of endogenous miR-375 function enhanced insulin secretion (Poy et al., Nature 432(7014); 226-30, 2004). MiR-375 was shown to target myotrophin (Mtpn) and inhibition of Mtpn mimicked the effects of miR-375 on glucose-stimulated insulin secretion and exocytosis.

The liver-specific miR-122 was inhibited in mice with a 2′-O-methoxyethyl phosphorothioate antisense oligonucleotide resulting in reduced plasma cholesterol levels, increased hepatic fatty-acid oxidation, and a decrease in hepatic fatty-acid and cholesterol synthesis rates (Esau et al., Cell Metab. 3(2):87-98, 2006). miR-122 inhibition in a diet-induced obesity mouse model resulted in decreased plasma cholesterol levels and a significant improvement in liver steatosis, accompanied by reductions in several lipogenic genes. These results implicate miR-122 as a key regulator of cholesterol and fatty-acid metabolism in the adult liver and suggest that miR-122 may be an attractive therapeutic target for metabolic disease.

miR-21 is exemplary of a miRNA with well-characterized targets and a function associated with the progression of diseases, such as cancer.

miR-21 is strongly overexpressed in glioblastoma and mir-21 knockdown in cultured glioblastoma cells, triggers activation of caspases and leads to increased apoptotic cell death (Chan et al., Cancer Res 66: 6029-6033, 2005). miR-21 is also up-regulated in breast cancer (lorio et al., Cancer Res 65: 7065-7070, 2005) and cholangiocarcinomas (Meng et al., Gastroenterology 130: 2113-2129, 2006).

Previously, identified targets for miR-21 include the tumor suppressors Tropomysin 1 (TPM1) in breast cancer cells (Zhu et al., J Biol Chem 282(19); 14328-14336, 2007) and Phosphatase and tensin homolog (PTEN) in hepatocellular carcinomas (Meng et al., Gastroenterology 133(2); 647-658, 2007). Given the importance of PTEN in regulating the Phosphoinositide Kinase 3/AKT pathway and the frequency of PTEN mutations or silencing in a variety of cancers this constitutes an appealing explanation for the over-expression of miR-21 observed in many cancer types.

Recently, PDCD4, CDK6, and Cofilin2 were shown to be directly regulated by mir-21 (Frankel et al., J Biol Chem in press; 1-10, 2007). PDCD4 is another tumor suppressor known to be upregulated during apoptosis and downregulated in several cancer forms such as lung cancer and hepatocellular carcinoma. We show herein that the miR-21 interaction with the miR-21 target site in the PDCD4 transcript can be inhibited by nucleic acids according to the present invention, providing specific inhibition of a miRNA target gene involved in disease.

An example of a miRNA that is frequently deregulated in cancer and target oncogenes is the let-7 miRNA family. lethal-7 (let-7) is temporally regulated in C. elegans, Drosophila, and zebrafish (Pasquinelli et al., Nature 408; 86-89, 2000; Reinhart et al., Nature 403; 901-906, 2000). In humans the let-7 family includes 12 homologues that have been found to map to regions deleted in human cancers (Calin et al., PNAS101; 2999-3004, 2004) and let-7 is poorly expressed in lung cancers (Takamizawa et al., Cancer Res 64; 3753-3756, 2004).

All three human RAS oncogenes have let-7 complementary sites in their 3′UTR and the let-7 miRNA family was shown to directly regulate the RAS oncogene negatively (Johnson et al., Cell 120: 635-647, 2005) implicating a function for let-7 as a tumor suppressor in lung tissue.

Another target of let-7, HMGA2, a high-mobility group protein, is oncogenic in a variety of tumors, including benign mesenchymal tumors and lung cancers. HMGA2 was derepressed upon inhibition of let-7 in cells with high levels of the miRNA. Ectopic expression of let-7 reduced HMGA2 and cell proliferation in a lung cancer cell. The effect of let-7 on HMGA2 was dependent on multiple target sites in the 3′ untranslated region (UTR), and the growth-suppressive effect of let-7 on lung cancer cells was rescued by overexpression of the HMGA2 ORF without a 3′UTR (Lee and Dufta, Genes & Development 21(9); 1025-1030, 2007).

In silico analysis of Disabled2 (Dab2), a putative tumor suppressor protein, 3′ UTR revealed miRNA complimentary to this region of the gene, suggesting that miRNA mediated targeting of Dab2 mRNA might account for loss of the protein in breast cancer (Bagadi et al., Breast Cancer Research and Treatment 104(3); 277-286.

Estrogen receptor α (ERα) is a target of miR-206 in breast cancer cell lines (Adams et al., Molecular Endocrinology 21(5); 1132-1147, 2007).

Cases of chronic lymphocytic leukaemia (CLL) with good prognostic features typically are characterized down-regulation of genes miR-15a and miR-16-1, located at 13q14.3. Both miRNAs negatively regulate BCL2 at a post-transcriptional level. On the other hand, in CLL cases that use unmutated Ig heavy-chain variableregion genes (IgVH) or have high level expression of the 70-kD zeta-associated protein (ZAP-70) have high levels of TCL1 due to low-level expression of miR-29 and miR-181, which directly target this oncogene (Calin et al. Best Practice & Research, Clinical Hematology 20(3); 425-437, 2007).

Felli et al. (PNAS 102; 18081-18086, 2005) described the ability of miR-221 and miR-222 to downregulate the KIT oncogene to modulate erythropoiesis in CD34+ hematopoiteic progenitor cells and inhibit cell growth of TF1 erythroleukemic cell line. He et al. (PNAS 102; 19075-19080, 2005) found that patients with papillary thyroid carcinomas showed decreased KIT transcript and protein levels and reciprocally increased levels of miR-221, miR-222 and miR-146 in the tumours.

Galardi et al. (JBC 282(32); 23716-23724) present further indications that miR-221/222 can be regarded as a new family of oncogenes, directly targeting the tumor suppressor p27Kip1, and that their overexpression might be one of the factors contributing to the oncogenesis and progression of prostate carcinoma through p27Kip1 down-regulation.

mir-122a modulates cyclin G1 expression in hepatocellular carcinoma (HCC) derived cell lines and an inverse correlation between miR-122a and cyclin G1 expression exists in primary liver carcinomas (Gramantieri et al., Cancer Research 67(13); 6092-6099, 2007).

miRNAs are aberrantly expressed in the vascular cell walls after balloon injury (Ji et al., Circulation Research 100 (11); 1579-1588, 2007) and knock-down of miR-21 had a significant negative effect on neointimal lesion formation. Western blot analysis demonstrated that PTEN and Bcl-2 were involved in miR-21 mediated cellular effects. The results suggest that miRNAs may be a new therapeutic target for proliferative vascular diseases such as atherosclerosis, postangioplasty restenosis, transplantation arteriopathy, and stroke.

Lecellier et al. (Science 308; 557-560, 2005) demonstrated that a mammalian miRNA, miR-32 restricts the accumulation of the retrovirus primate foamy virus type 1 (PFV-1) in human cells throwing light into the role of miRNAs in antiviral defense.

Computational methods incorporating both consensus prediction and target accessibility, have shown that human encoded miRNAs could target critical genes involved in the pathogenesis and tropism of influenza virus A/H5N1

WO2007042899 relates to the mapping of human miRNA targets in HIV genome including the nef gene which plays an important role in delayed disease progression (Hariharan et al., Biochem Biophys Res Commun 337(4):1214-8, 2005).

Stern-Ginossar et al. (Science 317(5836): 376-81, 2007) identified the major histocompatibility complex class I-related chain B (MICB) gene as a top candidate target of hcmv-miR-UL112, a human cytomegalovirus miRNA. MICB is a stress-induced ligand of the natural killer (NK) cell activating receptor NKG2D and is critical for the NK cell killing of virus-infected cells and tumor cells. It was shown that hcmv-miR-UL112 specifically down-regulates MICB expression during viral infection, leading to decreased binding of NKG2D and reduced killing by NK cells. The results reveal a miRNA-based immunoevasion mechanism that appears to be exploited by human cytomegalovirus.

Jopling et al. (Science 309; 1577-1581, 2005) reported a case wherein a liver specific miRNA miR-122 was shown to cause accumulation of viral RNA by binding to the 5′ non-coding region of the viral genome of hepatitis C virus.

Both computational tools and experimental validation of the predicted candidates have shown that viruses also encode miRNAs. Viruses with miRNA mediated regulation include herpesvirus, HIV and Simian Virus 40. Bennasser et al. (Retrovirology 1; 43, 2004) reported a computational screen for HIV-1 encoded miRNAs and further went about predicting their cellular targets and found five pre-miRNA candidates which has potential to encode 10 miRNAs and through them regulate ˜1000 host transcripts.

Of late, Cui et al. (J Virol 80; 5499-5508, 2006) discovered novel virus encoded miRNAs from HSV genome and Gupta et al. (Nature 442(7098):82-5, 2006) discovered that Herpes simplex-1 (HSV-1) latency associated transcript (LAT) encodes for a miRNA which target critical genes of the apoptosis pathway including TGF-β1 and SMAD3.

During the investigation of the function of miR-124 during spinal cord development, two endogenous targets of miR-124, laminγ1 and integrinβ1 were identified (Cao et al., Genes & Development 21(5); 531-536, 2007), both of which are highly expressed by neural progenitors but repressed upon neuronal differentation.

Kato et al. (PNAS 104(9); 3432-3437, 2007) uncovered a role for miR5 in the kidney and diabetic nephropathy in controlling TGFβ-induced collagen1α1 and -2 expression by down-regulating E-box repressors. Smad-interacting protein 1 (SIP1), a E-box repressor, was shown to be a target of miR-192, a key miR highly expressed in the kidney, in mouse mesangial cells.

HMGA2 expression has been shown to be associated with enhanced selective chemosensitivity towards the topoisomerase II inhibitor, doxorubicin in cancer cells. Herbert et al (Molecular Cancer, 6, 2007) report that HMGA2 expression in head and neck squamous cell carcinoma cells is regulated in part by miR-98. Transfection of pre-miR-98 during normoxia diminishes HMGA2 and potentiates resistance to doxorubicin and cisplatin.

ZFHX1B is a transcriptional repressor involved in the TGFbeta signaling pathway and in processes of epithelial to mesenchymal transition via regulation of E-cadherin. It was shown that Zfhx1b and miR-200b are regionally coexpressed in the adult mouse brain and that miR-200b represses the expression of Zfhx1b via multiple sequence elements present in the 3′-untranslated region. Overexpression of miR-200b leads to repression of endogenous ZFHX1B, and inhibition of miR-200b relieves the repression of ZFHX1B (Christoffersen et al., RNA 13(8); 1172-1178, 2007).

Co-expression of the miR-17-92 cluster acted with c-myc expression to accelerate tumour development in a mouse B-cell lymphoma model (He et al., Nature 435; 828-833, 2005).

The E2F family of transcription factors is essential in the regulation of the cell cycle and apoptosis. E2F1 appears to be negatively regulated by miR-17-5p and miR-20a, two members of the mir-17-92 cluster (Lewis et al, Cell 115(7); 787-798, 2003).

miR-20a also modulates the translation of the E2F2 and E2F3 mRNAs via binding sites in their 3′UTR (Sylvestre et al., JBC 282(4); 2135-2143, 2007). Overexpression of miR-20a decreased apoptosis in a prostate cancer cell line pointing toward an anti-apoptotic role for miR-20a. In addition evidence suggesting an autoregulatory feedback loop between E2F factors and miRNAs from the mir-17-92 cluster has been presented.

The mir-17-92 cluster is overexpressed in lung cancers, especially in the most aggressive small-cell lung cancer (Hayashita et al., Cancer Res. 65; 9628-9632, 2005).

Ozen et al. (Oncogene, Sep. 24, 2007, Epub ahead of print) found and verified widespread, but not universal, downregulation of miRNAs in clinically localized prostate cancer relative to benign peripheral zone tissue. The down-regulated miRNAs include several with proven target mRNAs whose proteins have been previously shown to be increased in prostate cancer by immunohistochemistry, including RAS, E2F3, BCL-2 and MCL-1. Using a bioinformatics approach, they identified additional potential mRNA targets of one of the miRNAs, (miR-125b) that are upregulated in prostate cancer and confirmed increased expression of one of these targets, EIF4EBP1, in prostate cancer tissues.

The heart responds to diverse forms of stress by hypertrophic growth accompanied by fibrosis and eventual diminution of contractility, which results from down-regulation of α-myosin heavy chain (αMHC) and up-regulation of βMHC, the primary contractile proteins of the heart.

The cardiac-specific miRNA, miR-208, encoded by an intron of the αMHC gene was found to be required for cardiomyocyte hypertrophy, fibrosis and expression of βMHC in response to stress and hypothyroidism in miR-208 mutant mice (Rooji et al., Science 316; 575-579, 2007). Experiments indicated that a predicted miR-208 target, thyroid hormone receptor associated protein 1 (THRAP1), is negatively regulated by miR-208 implicating that miR-208 acts, at least in part, by repressing expression of the thyroid hormone receptor coregulator THRAP1, which can exert positive and negative effects on transcription.

Welch et al. (Oncogene 26(34): 5017-5022, 2007) has shown that miR-34a is generally expressed at lower levels in unfavorable primary neuroblastoma (NB) tumors and cell lines relative to normal adrenal tissue and that reintroduction of this miRNA into three different NB cell lines causes a dramatic reduction in cell proliferation through the induction of a caspase-dependent apoptotic pathway. As a potential mechanistic explanation for this observation, they also demonstrated that miR-34a directly targets the mRNA encoding E2F3 and significantly reduces the levels of E2F3 protein, a potent transcriptional inducer of cell-cycle progression. Furthermore, miR-34a expression increases during retinoic acid-induced differentiation of the SK-N-BE cell line, whereas E2F3 protein levels decrease. Thus, adding to the increasing role of miRNAs in cancer, miR-34a may act as a suppressor of NB tumorgenesis.

Using a miRNA microarrays platform and quantitative real time-polymerase chain reaction, miR-15a, miR-15b, miR-16-1, let-7a-3, let-7c, let-7d, miR-223, miR-342 and miR-107 was found to be upregulated in acute promyelocytic leukaemia patients and cell lines during all-trans-retinoic acid (ATRA) treatment, whereas miR-181b was downregulated (Garzon et al., Oncogene 26(28): 4148-4157, 2007). miR-107 was verified to target NFI-A, a gene that has been involved in a regulatory loop involving miR-223 and C/EBPa during granulocytic differentiation and ATRA down-regulation of RAS and Bcl2 correlated with the activation of known miRNA regulators of those proteins, let-7a and miR-15a/miR-16-1, respectively.

Deregulation of the TCL1 oncogene is a causal event in the pathogenesis of the aggressive form B-cell chronic lymphocytic leukemia (B-CLL). Pekarsky et al. (Cancer Res. 66(24); 11590-11593, 2006) demonstrated that Tcl1 expression is regulated by miR-29 and miR-181, two miRNAs differentially expressed in CLL. Expression levels of miR-29 and miR-181 generally inversely correlated with Tcl1 expression in the examined CLL samples.

miRNA also target Cytochrome P450 (CYP), a superfamily of drugmetabolizing enzymes. Human CYP1B1, which is highly expressed in estrogen target tissues, catalyzes the metabolic activation of various procarcinogens and the 4-hydroxylation of 17beta-estradiol and was shown to be post-transcriptionally regulated by miR-27b (Tsuchiya et al., Cancer Res. 66(18); 9090-9098, 2006). In most breast cancer patients, the expression level of miR-27b was decreased in cancerous tissues, accompanied by a high level of CYP1B1 protein. A significant inverse association was observed between the expression levels of miR-27b and CYP1B1 protein.

Hossain et al. (Mol. Cell. Biol. 26(21); 8191-201, 2006) reported a role for miR-17-5p as a tumor suppressor in breast cancer cells.

miR-17-5p has extensive complementarity to the mRNA of AIB1 (amplified in breast cancer 1). Cell culture experiments showed that AIB1 expression was downregulated by miR-17-5p, primarily through translational inhibition and that down-regulation of AIB1 by Mir-17-5p resulted in decreased estrogen receptor-mediated, as well as estrogen receptor-independent, gene expression and decreased proliferation of breast cancer cells. miR-17-5p also completely abrogated the insulin-like growth factor 1-mediated, anchorage-independent growth of breast cancer cells.

In non-cell-autonomous Myc-induced tumor phenotypes, miRNAs have been shown to have a role involving the regulation of anti-angiogenic thrombospondin-1 (Tsp1) and connective tissue growth factor (CTGF), which are targets for repression by the miR-17-92 cluster, which is upregulated in colonic epithelial cells coexpressing K-Ras and c-Myc (Dews et al., Nature Genetics 38(9); 1060-1065, 2006).

Some patients who suffer from Tourettes' syndrome, a neuropsychiatric disorder characterized by persistent vocal and motor tics, have mutations in the miR-189 target site within the 3′ UTR of the gene encoding SLITRK1, a single pass transmembrane protein with a leucine-rich extracellular domain (Abelson et al., Science 310, 317-320, 2005), providing an example that mutations in the 3′ UTR of the candidate disease genes that disrupt specific miRNA binding sites can impact diseases through reduced or total loss of miRNA-mediated regulation.

### Pharmaceutical Compositions

The oligos of the invention may be formulated into pharmaceutical compositions for administration to human subjects in a biologically compatible form suitable for administration in vivo.

The oligos of the invention may be used in the form of the free acid, free base, in the form of salts, solvates, and as prodrugs. All forms are within the scope of the invention. In accordance with the methods of the invention, the described oligos or salts, solvates, or prodrugs thereof may be administered to a patient in a variety of forms depending on the selected route of administration, as will be understood by those skilled in the art. The oligos of the invention may be administered, for example, by oral, parenteral, buccal, sublingual, nasal, rectal, patch, pump, or trans-dermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

An oligo of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, an oligo of the invention may be incorporated with an excipient and used in the form of ingestible tablets, buccal tablets, troches, capsules, elixirs, suspensions, syrups, wafers, and the like.

An oligo of the invention may also be administered parenterally. Solutions of an oligo of the invention can be prepared in water suitably mixed with a surfactant, such as hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003—20th edition) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pumpatomizer.

Compositions suitable for buccal or sublingual administration include tablets, lozenges, and pastilles, where the active ingredient is formulated with a carrier, such as sugar, acacia, tragacanth, or gelatin and glycerine. Compositions for rectal administration are conveniently in the form of suppositories containing a conventional suppository base, such as cocoa butter.

The oligos of the invention may be administered to an animal alone or in combination with pharmaceutically acceptable carriers, as noted above, the proportion of which is determined by the solubility and chemical nature of the compound, chosen route of administration, and standard pharmaceutical practice.

The dosage of the oligos of the invention, and/or compositions comprising an oligo of the invention, can vary depending on many factors, such as the pharmacodynamic properties of the oligo; the mode of administration; the age, health, and weight of the recipient; the nature and extent of the symptoms; the frequency of the treatment, and the type of concurrent treatment, if any; and the clearance rate of the oligo in the animal to be treated. One of skill in the art can determine the appropriate dosage based on the above factors. The oligos of the invention may be administered initially in a suitable dosage that may be adjusted as required, depending on the clinical response.

In addition to the above-mentioned therapeutic uses, an oligo of the invention can also be used in diagnostic assays, screening assays, and as a research tool.

In diagnostic assays, an oligo of the invention may be useful in identifying or detecting a particular miRNA target sequence. For such a use, the oligo may be labelled, e.g., fluorescently labelled or radiolabelled, and contacted with a population of cells of an organism or a nucleic acid sample from an organism.

As another example, an isolated sample from a patient could be cultured ex vivo, and a blocking nucleic acid of the invention may be administered to the sample to modulate the interaction between a specific nucleic acid target (such as a mRNA) and a miRNA. In one application, a proposed treatment could be co-administered to test if modulating a specific miRNA would render the disease more or less sensitive to such proposed treatment.

The present oligonucleotides of the invention are furthermore useful and applicable for large-scale and genome-wide expression profiling of nucleotide targets to determine the prevalence of specific miRNA target site containing nucleotides by oligonucleotide microarrays.

In screening assays, an oligo of the invention may be used to identify other compounds that prevent an miRNA from binding to a particular target site. As research tools, the oligos of the invention may be used in enzyme assays and assays to study the localization of miRNA activity. Such information may be useful, for example, for diagnosing or monitoring disease states or progression. In such assays, an oligo of the invention may also be labelled.

### EXAMPLES

The invention will now be further illustrated with reference to the following examples. It will be appreciated that what follows is by way of example only and that modifications to detail may be made while still falling within the scope of the invention.

### Example 1

### Synthesis, Deprotection and Purification of LNA-Substituted Oligonucleotides

LNA-substituted oligos were prepared on an automated DNA synthesizer (Expedite 8909 DNA synthesizer, PerSeptive Biosystems, 0.2 μmol scale) using the phosphoramidite approach (Beaucage and Caruthers, Tetrahedron Leff. 22: 1859-1862, 1981) with 2-cyanoethyl protected LNA and DNA phosphoramidites, (Sinha, et al., Tetrahedron Lett. 24: 5843-5846, 1983). CPG solid supports derivatised with a suitable quencher and 5′-fluorescein phosphoramidite (GLEN Research, Sterling, Va., USA). The synthesis cycle was modified for LNA phosphoramidites (250 s coupling time) compared to DNA phosphoramidites. 1H-tetrazole or 4,5-dicyanoimidazole (Proligo, Hamburg, Germany) was used as activator in the coupling step.

The probes were deprotected using 32% aqueous ammonia (1 h at room temperature, then 2 hours at 60° C.) and purified by HPLC (Shimadzu-SpectraChrom series; Xterra™ RP18 column, 10 μM 7.8×150 mm (Waters). Buffers: A: 0.05M Triethylammonium acetate pH 7.4. B. 50% acetonitrile in water. Eluent: 0-25 min: 10-80% B; 25-30 min: 80% B). The composition and purity of the probes were verified by MALDI-MS (PerSeptive Biosystem, Voyager DE-PRO) analysis.

### Example 2

### Design of Blocking Molecules

Previous experiments using antagonizing oligos have demonstrated that important parameters for successful design are probe Tm and oligo self-annealling. If oligonucleotide Tm is too low, the efficiency is generally poor, maybe due to the oligo being removed from the target sequence by endogenous helicases. If Tm is too high, there is an increased risk that the oligo will anneal to partly complementary sites possibly leading to unspecific effects, known as off-targets effects. With respect to selfannealing (autocomplementarity) of the probe, a low selfannealing score (reflecting stability of the autoduplex) is favorable. Previous results have shown that probes exceeding a selfannealing score of about 45 often show very low potency or are completely nonfunctional. The effect of a high selfannealing score is a stable autoduplex which obviously sequestrates large amounts of probes, preventing the probe from interacting with its target sequence. To avoid high stability of the autoduplex, it is important to prevent LNA nucleotides in stretches of autocomplementary sequences. This may be achieved by an iterative approach in which the starting point is an oligonucleotide sequence consisting of only LNA monomers. This oligonucleotide is then put through a selfannealing scoring program (Exiqon website) that also identifies nucleotides participating in duplex formation. Next, one or more of these nucleotides are substituted with DNA, and the process is repeated, again substituting LNAs participating in duplex formation with DNA thereby gradually reducing selfannealing score. When reaching an appropriate Tm and selfannealing score, the process is stopped. The process is repeated for sequences spanning various regions of the target sequence to find optimal selfannealing scores and Tms.

An additional requirement in an oligonucleotide design process was the preference of LNA nucleotides in the terminals of the oligonucleotide. This was done to preserve biostability of the oligo, thereby improving duration of the biological response.

All references, patents, and patent applications cited herein are hereby incorporated by reference.

Other embodiments are in the claims.

What is claimed is:

1. A method of identifying the base succession of a naturally occurring nucleotide sequence involved in regulating the activity of a miRNA comprising hybridizing one oligonucleotide to a naturally occurring nucleotide sequence downstream or upstream of a target site of a miRNA, wherein said hybridizing identifies the base succession of a naturally occurring nucleotide sequence involved in regulating the activity of said miRNA.

2. A method of identifying or verifying the presence of one or more naturally occurring nucleotide sequence(s) involved in regulating the activity of a miRNA comprising
a) contacting a nucleic acid sample from a subject with an oligonucleotide hybridizing to a naturally occurring nucleotide sequence downstream or upstream of a target site of said miRNA, and
b) determining the activity of said miRNA in said nucleic acid sample, wherein a change in the activity of said miRNA identifies said naturally occurring nucleotide sequence as being involved in regulating the activity of said miRNA.

3. The method of claim 2, comprising repeating step a) and b) one or more time(s), each time using an oligonucleotide hybridizing to a different or overlapping, naturally occurring nucleotide sequence downstream or upstream of a target site of said miRNA.

4. The method of claim 2, wherein said at least one oligonucleotide comprises at least one high affinity nucleic acid analog.

5. The method of claim 4, wherein said at least one high affinity nucleic acid analog is LNA.

6. The method of claim 2, wherein said contacting occurs in a cell.

7. The method of claim 6, wherein said cell expresses said miRNA.

8. The method of claim 2, wherein the activity of said miRNA is the binding activity of said miRNA to said target site.

9. A nucleic acid compound comprising at least one region hybridizing to a naturally occurring nucleotide sequence of 5-30 nucleotides located downstream or upstream of a target site of a miRNA, wherein said nucleic acid compound does not hybridize to said target site but is capable of inhibiting the binding of said miRNA to said target site.

10. The nucleic acid compound according to claim 9, wherein said naturally occurring nucleotide sequence is located 1-500 nucleotides downstream or upstream of said target site.

11. The nucleic acid compound according to claim 10 comprising at least one high affinity nucleic acid analog.

12. The nucleic acid compound according to claim 11, wherein said at least one high affinity nucleic acid analog is LNA.

13. The nucleic acid compound of claim 9, wherein said naturally occurring nucleotide sequence is located adjacent to said target site.

14. The nucleic acid compound of claim 9, wherein said naturally occurring nucleotide sequence comprises a naturally occurring nucleotide sequence involved in regulating the activity of said miRNA.

15. The nucleic acid compound of claim 14, wherein said naturally occurring nucleotide sequence involved in regulating the activity of said miRNA is identified by the method of claim 2.

16. The nucleic acid compound of claim 13 further comprising a region non-complementary to said target site and overlapping said target site.

17. The nucleic acid compound of according claim 13 further comprising a blocking moiety overlapping said target site.

18. The nucleic acid compound of claim 9 comprising a first region hybridizing to a naturally occurring nucleotide sequence downstream of said target site and a second region hybridizing to a naturally occurring nucleotide sequence upstream of said target site.

19. The nucleic acid compound of claim 18 comprising a linker between said first region and said second region, wherein said linker is non-complementary to said target site.

20. The nucleic acid compound of claim 19, wherein said linker comprises a nucleic acid sequence of between 2-30 nucleotides.

21. The nucleic acid compound of claim 18, wherein said linker comprises an alkyl chain.

22. The nucleic acid compound of claim 9, wherein hybridization of said at least one region to said naturally occurring nucleotide sequence reduces the binding of said miRNA to said target site.

23. The nucleic acid compound of claim 22, wherein hybridization of said at least one region to said naturally occurring nucleotide sequence reduces the binding of said miRNA to said target site by at least 50%.

24. The nucleic acid compound of claim 9, wherein said nucleic acid compound is RNase resistant.

25. The nucleic acid compound of claim 9, wherein said nucleic acid compound comprises up to 80% of said at least one high affinity nucleic acid analog or said at least one high affinity nucleic acid analog in combination with one or more additional analogs.

26. The nucleic acid compound of claim 9, wherein said at least one region hybridizes to said nucleotide sequence with a Kd lower than the Kd whereby said miRNA binds to said target site.

27. The nucleic acid compound of claim 9, wherein said at least one region has an increase in binding affinity to said naturally occurring nucleotide sequence determined by an increase in Tm of at least 2° C., compared to the naturally occurring RNA complement of said nucleotide sequence.

28. The nucleic acid compound of claim 9, wherein said miRNA is associated with cancer, heart disease, cardiovascular disease, neurological disease, atherosclerosis, postangioplasty restenosis, transplantation arteriopathy, stroke, infection, hepatitis C, HIV, psoriasis, metabolic disease, diabetes mellitus, or diabetic nephropathy.

29. A pharmaceutical composition comprising one or more nucleic acid compound(s) of claim 9 and a pharmaceutically acceptable excipient.

30. (canceled)

31. A method of inhibiting the binding of a miRNA to a target site, said method comprising administering one or more nucleic acid compound(s) of claim 9 to a cell expressing said target site.

32. (canceled)

33. (canceled)

34. A method of treating a disease said method comprising administering to a subject one or more nucleic acid compound(s) of claim 9 in an amount sufficient to reduce the activity of a miRNA associated with said disease.

35. The method according to claim 34, wherein said disease is selected from the group comprising cancer, heart disease, cardiovascular disease, neurological disease, atherosclerosis, postangioplasty restenosis, transplantation arteriopathy, stroke, infection, hepatitis C, HIV, psoriasis, metabolic disease, diabetes mellitus, and diabetic nephropathy.
